(19) United States
(12) Patent Application Publication
(10) Pub. No.: US 2003/0100501 A1
(43) Pub. Date: May 29, 2003

(54) Q4N2NEG2 enhances CFTR activity

(75) Inventors: Pamela Bowes Davis, Cleveland Heights, OH (US); Jianjie Ma, Belle Mead, NJ (US); Thomas Gerken, Solon, OH (US)

Correspondence Address: BANNER & WITCOFF, 1001 G STREET N W, SUITE 1100, WASHINGTON, DC, 20001, US

(73) Assignee: Case Western Reserve University, Cleveland, OH (US)

(21) Appl. No.: 10/252,012

(22) Filed: Sep. 23, 2002

Related U.S. Application Data
(60) Provisional application No. 60/323,724, filed on Sep. 21, 2001.

(51) Int. Cl. A61K 38/17; C07K 14/705

(52) U.S. Cl. 514/12; 530/350

(57) ABSTRACT

Phosphorylation of the cystic fibrosis transmembrane conductance regulator (CFTR) by cyclic AMP-dependent protein kinase (PKA) is essential for opening the CFTR chloride channel. A short segment containing many negatively charged amino acids (817-838, NEG2) within the regulatory (R) domain of CFTR is a critical regulator of the chloride channel activity. An isolated NEG2 polypeptide may be expressed as a separate sequence that stimulates CFTR channel openings at lower concentrations, but that inhibits CFTR channel openings at higher concentrations. Residues in the NEG2 sequence were substituted to produce a polypeptide that exerts only an activating effect on CFTR. One such polypeptide is the Q4N2NEG2 polypeptide. Exogenous Q4N2NEG2 exerts stimulatory effects on both wild-type and mutant G551D CFTR function, without exhibiting inhibitory activity at any concentration.

This application is a non-provisional of and claims priority to U.S. Provisional Application Serial No. 60/323,724, filed Sep. 21, 2001, the disclosure of which is expressly incorporated herein.

This invention was made with government support under RO1 HL/DK 49003, P30 DK27651 and RO1 DK51770 awarded by the National Institute of Health. The government has certain rights in the invention

### TECHNICAL FIELD OF THE INVENTION

This invention is related to the field of cystic fibrosis. More particularly, it is related to the area of therapeutic treatments and drug discovery for treating cystic fibrosis.

### BACKGROUND OF THE INVENTION

Defects in CFTR, a chloride channel located in the apical membrane of epithelial cells, are associated with the common genetic disease, cystic fibrosis (Quinton, 1986, Welsh and Smith, 1993, Zielenski and Tsui, 1995). CFTR is a 1480 amino acid protein that is a member of the ATP binding cassette (ABC) transporter family (Riordan et al., 1989, Higgins, 1992). Each half of CFTR contains a transmembrane domain and a nucleotide binding fold (NBF), and the two halves are connected by a regulatory, or R domain. The R domain is unique to CFTR and contains several consensus PKA phosphorylation sites (Cheng et al., 1991, Picciotto et al., 1992). Opening of the CFTR channel is controlled by PKA phosphorylation of serine residues in the R domain (Tabcharani et al., 1991, Bear et al., 1992) and ATP binding and hydrolysis at the NBFs (Anderson et al., 1991, Gunderson and Kopito, 1995). Phosphorylation adds negative charges to the R domain, and introduces global conformational changes reflected by the reduction in the α-helical content of the R domain protein (Dulhanty and Riordan, 1994). Thus, electrostatic and/or allosteric changes mediated by phosphorylation are likely to be responsible for interactions between the R domain and other CFTR domains that regulate channel function (Rich et al., 1993, Gadsby and Naim, 1994).

Rich et al., 1991 showed that deletion of amino acids 708-835 from the R domain (ΔR-CFTR), which removes most of the PKA consensus sites, renders the CFTR channel PKA independent, but the open probability of ΔR-CFTR is one-third that of the wild type channel and does not increase upon PKA phosphorylation (Ma et al., 1997, Winter and Welsh, 1997). Thus, it is possible that deletion of the R domain removes both inhibitory and stimulatory effects conferred by the R domain on CFTR chloride channel function. This conclusion is supported by studies that show that addition of exogenous unphosphorylated R domain protein (amino acids 588-858) to wt-CFTR blocks the chloride channel (Ma et al., 1996), suggesting that the unphosphorylated R domain is inhibitory. Conversely, exogenous phosphorylated R domain protein (amino acids 588-855 or 645-834) stimulated the ΔR-CFTR channel, suggesting that the phosphorylated R domain is stimulatory (Ma et al., 1997, Winter and Welsh, 1997). Therefore, it appears that the manifest activity (stimulatory or inhibitory) depends on the phosphorylation state of the R domain.

About 25% of the known 700 mutations in CFTR produce a mutant CFTR protein which is properly transported to the apical membrane of epithelial cells but have only low level, residual channel activity. There is a need in the art for agents which can boost the level of channel activity in those mutants having low level activity.

### SUMMARY OF THE INVENTION

These and other objects of the invention are achieved by providing one or more of the embodiments described below. In one embodiment of the invention an isolated polypeptide is provided. The polypeptide comprises an amino acid sequence of SEQ ID NO: 6 wherein the polypeptide retains a net negative charge of 1-8. More preferably the variant of said CFTR protein has the sequence of SEQ ID NO: 1.

In another embodiment of the invention a method is provided for activating a CFTR protein. An effective amount of the polypeptide is administered to a cell comprising a CFTR protein that forms a cAMP regulated chloride channel. The polypeptide comprises the sequence of SEQ ID NO: 6. The CFTR protein is consequently activated. More preferably, the polypeptide has the sequence of SEQ ID NO: 1.

According to another embodiment of the invention a method is provided for activating a CFTR protein. An effective amount of a polypeptide is contacted with a CFTR protein in a lipid bilayer wherein the polypeptide comprises the amino acid sequence of SEQ ID NO: 6. The CFTR protein is thereby activated. More preferably, the polypeptide comprises the amino acid sequence of SEQ ID NO: 1.

In another embodiment of the invention a method is provided for synthesizing a CFTR-related polypeptide. Units of one or more amino acid residues are linked to form a polypeptide comprising the amino acid sequence of SEQ ID NO: 6. More preferably, the polypeptide has the sequence of SEQ ID NO: 1.

In another embodiment of the invention an isolated polypeptide is provided. The polypeptide comprises the amino acid sequence of SEQ ID NO: 2.

In yet another embodiment of the invention a nucleic acid molecule is provided. The nucleic acid comprises a nucleotide sequence encoding a polypeptide according to SEQ ID NO: 2.

In another embodiment of the invention a method of activating a CFTR protein is provided. A nucleic acid comprising a sequence encoding a polypeptide according to SEQ ID NO: 2 is administered to a cell comprising the CFTR protein, whereby the polypeptide is expressed and the CFTR protein is activated.

These and other embodiments of the invention, which will be apparent to those of skill in the art, provide the art with reagents and tools for enhancing function of cAMP regulated chloride channels that are defective in cystic fibrosis patients.

### BRIEF DESCRIPTION OF THE DRAWINGS

FIG. 1A and 1B and 1C: Demonstration of increase in open probability of CFTR channel with addition of the Q4 N2 NEG2 peptide.

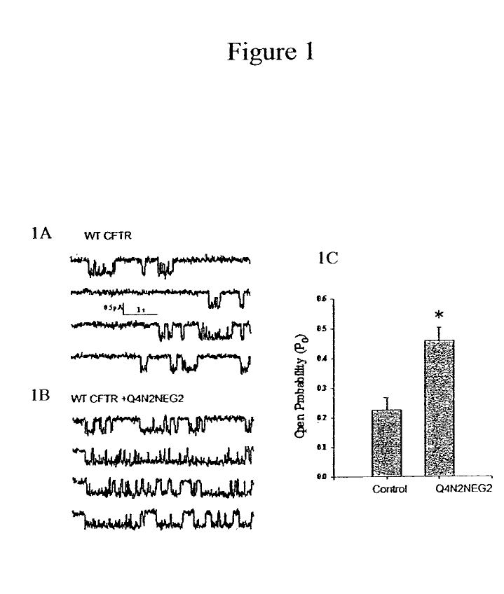

(FIG. 1A) Single channel trace of the CFTR channel before addition of peptide.

(FIG. 1B) Single channel trace after addition of Q4 N2 NEG2 peptide (4 μM).

(FIG. 1C) Summary of five separate experiments. Addition of Q4N2 NEG2 peptide increases the Po by about two-fold.

### DETAILED DESCRIPTION OF THE INVENTION

It is a discovery of the present inventors that the channel inhibitory properties of the R domain of CFTR protein can be separated from the channel activating properties. Thus activating polypeptides can be used to treat CFTR defective cells, without concern for inhibition at certain concentrations. Activating polypeptides may also be used to enhance the activity of normal CFTR, including that delivered by gene transfer.

A polypeptide for use in treating CFTR-defective cells contains a 22 amino acid sequence, GLXISXXINXXXLKXXFFXXXX, as shown in SEQ ID NO: 6. The amino terminal residue is acetylated and the carboxy terminal residue is amidated. The residue X, at positions 3, 6, 7, 10, and 11 is either glutamic acid or glutamine; at position 12 is aspartic acid or asparagine; at position 15 is glutamic acid or glutamine; at position 16 is cysteine or serine; at positions 19 or 20 is aspartic acid or asparagine; at position 21 is methionine or norleucine; at position 22 is either glutamic acid or glutamine. The amino acid residue at position 16 is more preferably serine. The amino residue at position 21 is more preferable norleucine. The polypeptide of SEQ ID NO: 6 has a net negative charge. The net negative charge is preferably within the ranges of 1-8, 2-8, 3-8, 4-8, 5-8, 6-8, or 7-8.

The polypeptide more preferably has the sequence of SEQ ID NO: 1, GLEISEQINQQNLKQSFFNDLE, wherein L at position 21 is norleucine. The amino terminal residue of the polypeptide is preferably acetylated and the carboxy terminal residue is preferably amidated.

The polypeptide may also be present in a composition with a pharmaceutically acceptable carrier. Pharmaceutically acceptable carriers are well known to those in the art. Pharmaceutically acceptable carriers include, but are not limited to, large, slowly metabolized macromolecules, such as proteins, polysaccharides, polylactic acids, polyglycolic acids, polymeric amino acids, amino acid copolymers, and inactive virus particles. The composition can also contain liquids, such as water, saline, glycerol, and ethanol, as well as substances such as wetting agents, emulsifying agents, or pH buffering agents. Buffering agents include Hanks' solution, Ringer's solution, or physiologically buffered saline.

It may be desirable that the polypeptide be fused to another polypeptide to provide additional functional properties. For example, fusion to another protein such as keyhole limpet hemocyanin can be used to increase immunogenicity. Another desirable fusion partner is a membrane-penetrating peptide. Such peptides include VP-22 (SEQ ID NO: 3), as well as the peptides shown in SEQ ID NO: 4 and SEQ ID NO: 5. Such peptides can be used to facilitate the uptake of the polypeptide by target cells. The polypeptides of the invention may also be fused to proteins that cause specific targeting to lung epithelial cells. For instance, the peptide THALWHT directs DNA to human airway epithelial cells. Single chain antibody variable domains may be used to do the same.

A CFTR protein can be activated by the polypeptide. The CFTR protein can be in a cell, preferably in the cell membrane and the CFTR protein forms a cAMP-regulated chloride channel. An effective amount of a polypeptide that comprises the sequence of SEQ ID NO: 6 can be administered to the cell, and administration of the polypeptide activates the CFTR protein. The polypeptide administered more preferably comprises the sequence of SEQ ID NO: 1.

The cells may be any cells that contain or express a CFTR protein. The cells may naturally express the CFTR protein, such as lung epithelial cells, or the cells may express the CFTR protein after transient or stable transformation. The cells may be primary cells isolated from individuals that express a wild-type CFTR protein, or may be primary cells isolated from individuals that express a mutant CFTR protein. The cells may also be of a stable cell line. The cells may also exist in the body.

The CFTR protein is a wild type or a mutant CFTR protein. The mutant CFTR protein is a CFTR protein that is expressed by the cells and that is transported to the cell surface. The mutant CFTR protein also forms a cAMP-regulated chloride channel. The mutant CFTR protein may contain alterations that are known and characterized, or may contain alterations that have not yet been discovered. A mutant CFTR protein that fails to undergo full activation is a CFTR protein that does not conduct ions to the same degree as wild-type CFTR. The mutant CFTR protein may not conduct ions at all. The mutant protein may also conduct ions to a similar extent as wild type CFTR but be present in the membrane in substantially lower amounts than is true for normal individuals.

Activated is defined as any increase in conductance by the CFTR protein. An increase in conductance may result when the opening of the CFTR channel occurs with greater frequency than previously observed. An increase in CFTR conductance may result when the duration of opening is increased each time the CFTR channel opens. An increase in conductance may also result due to greater ability to conduct ions each time the CFTR protein channel is open. The increase in open probability of the CFTR protein is preferably at least 25%, at least 50%, at least 75%, at least 100%, at least 125%, at least 150%, at least 175%, at least 200%, or at least 300%.

An effective amount is any amount of polypeptide that is sufficient to activate the CFTR protein, as activate is defined above. Preferably, the polypeptide is administered to achieve a concentration of 0.5 to 14 μM. More preferably, the polypeptide is administered to achieve a concentration of 4-6 μM.

The polypeptide may be administered by any means acceptable in the art. For instance, the polypeptide may be administered in vitro, or to cells in culture, by addition to the medium. The polypeptide may be administered in vivo, to a patient, by any route including intravenous, intrathecal, oral, intranasal, transdermal, subcutaneous, intraperitoneal, parenteral, topical, sublingual, or rectal. Most preferably, the polypeptide is administered to a patient in an aerosol.

The aerosolized polypeptide can be co-administered with an expression vector that encodes wild type CFTR protein. An expression vector may be linear DNA that encodes wild type CFTR protein, or a plasmid or human artificial chromosome that expresses wild type CFTR protein. The vector may be administered as naked DNA or may be administered complexed to lipid molecules such as with liposomes, short polypeptides such as the THALWHT polypeptide, or polycations such as polylysine, with or without stabilizing agents and/or receptor ligands. The DNA may also be administered in a viral vector. Viral vectors are known in the art. Several nonlimiting examples include retroviruses, adenoviruses, adeno-associated viruses, lentiviruses, and herpes simplex virus. The gene encoding the wild type CFTR protein may additionally comprise a promoter sequence to drive expression of the CFTR gene. Any promoter known in the art may be used. Promoters include strong promoters such as the promoters of cytomegalovirus, SV40, or Rous sarcoma virus. The promoter may also be a tissue specific promoter. Preferably the tissue specific promoter is a lung specific promoter. Lung specific promoters include the promoters of surfactant protein A, keratin 18, Du Clara cell secretory protein, and the promoter of CFTR.

A CFTR protein can also be activated by applying an effective amount of a polypeptide to a CFTR protein in a lipid bilayer. The polypeptide comprises the amino acid sequence of SEQ ID NO: 6. The polypeptide more preferably comprises the amino acid sequence of SEQ ID NO: 1. Activating a CFTR protein in a lipid bilayer is useful to the art for screening agents for the treatment of cystic fibrosis.

A CFTR protein in a lipid bilayer may be a CFTR protein that is expressed in cells in culture. The cells may express the CFTR protein without manipulation, or may be stably or transiently transfected to express the CFTR protein. The lipid bilayer may also be such artificial preparations as, without limitation, a microsome preparation, a lipid-bilayer vesicle preparation, or liposomes. The polypeptide may be applied to the protein by its addition to cell culture media, or solution in which the lipid bilayers are maintained. A change in conductance may be measured by any means known in the art, such as patch clamping.

A CFTR activating polypeptide can be synthesized by sequentially linking units of one or more amino acid residues to form a polypeptide comprising the amino acid sequence of SEQ ID NO: 6. Preferably the polypeptide has the amino acid sequence of SEQ ID NO: 1. Synthesis of the CFTR polypeptide can be performed using solid-phase synthesis, liquid-phase synthesis, semisynthesis, or enzymatic synthesis techniques. Preferably the polypeptides are synthesized by solid-phase synthesis. More preferably the peptides are synthesized by F-moc synthesis.

The polypeptide of the invention may alternatively comprise the sequence of SEQ ID NO: 2, GLEISEQINQQNLKQSFFNDME. The polypeptide of SEQ ID NO: 2 is not modified. It is similar to the sequence of SEQ ID NO: 1, but for a methionine at position 21, rather than a norleucine. Like SEQ ID NO: 1 and SEQ ID NO: 6, it may be fused to a membrane penetrating polypeptide.

Nucleic acid molecules comprise a nucleotide sequence that encodes the polynucleotide sequence of SEQ ID NO: 2. One of skill in the art will recognize that many sequences will encode the polypeptide, as more than one codon can specify a given amino acid. The nucleic acid may further comprise regulatory sequences that enhance the expression of the polypeptide. Promoters may be strong constitutive promoters, as discussed above, or may be tissue-specific promoters. Preferably the tissue-specific promoter is a lung-specific promoter. The nucleic acid molecules may further comprise a vector. The vector can be any suitable vector for the delivery of the polynucleotide sequence into the lungs of a patient, resulting in expression of the polypeptide in the lungs of the patient.

A CFTR protein can be activated by expression of a polynucleotide. A nucleic acid comprising a sequence encoding a polypeptide according to SEQ ID NO: 2 is administered to a cell comprising the CFTR protein. The polypeptide is expressed and the CFTR protein is thereby activated. The polynucleotide may be administered by any acceptable means in the art. Preferably the polynucleotide is administered as an aerosol.

The administration of the polypeptides of the present invention are most useful in treatment of a class of mutations that encode CFTR proteins that are properly delivered to the plasma membrane but that are residually or minimally active. Minimally or residually active CFTR proteins have the ability to mediate or modulate channel conductance. However, channel conductance is insufficient to sustain the healthy, not cystic fibrotic phenotype. Residually or minimally active includes proteins for which the activity of the CFTR can be recorded but may be at a level that is barely detectable. This invention will also be useful for CFTR mutants that are, to a large extent, misprocessed and thus reach the plasma membrane in much lower quantities than normally processed CFTR, and for CFTR mutants that are, to a large extent, improperly spliced, but retain production of some properly spliced CFTR. Known mutants of CFTR are listed in Table 1. In addition to its utility in the activation of mutant forms of CFTR, this invention will be a useful adjunct to gene therapy for cystic fibrosis. By enhancing the per-CFTR molecule chloride transport activity, this peptide will increase the chloride transport activity obtained at any level of expression of CFTR, thereby increasing its effective efficacy.

TABLE 1

| Name | Nucleotide_change | Exon | Consequence | Reference |
|---|---|---|---|---|
| −816C − >T | C to T at −816 | 5′ | promoter mutation? | Bienvenu et al. (NL#60) |
| | | flanking | | |
| −741T − >G | T to G at −741 | 5′ | promoter mutation? | Bienvenu et al. (NL#59) |
| | | flanking | | |
| −471delAGG | deletion of AGG from −471 | 5′ | promoter mutation? | Grade et al. 1994 |
| | | flanking | | |
| −363C/T | C to T at −363 | 5′ | promoter mutation | Zielenski et al. 1999* |
| | | flanking | | |
| −102T − >A | T to A at −102 | 5′ | regulatory mutation? | Claustres et al. (NL#69) |
| | | flanking | | |
| −94G − >T | G to T at −94 | 5′ | promoter mutation? | Claustres et al. (NL#70) |
| | | flanking | | |
| −33G − >A | G to A at −33 | 5′ | promoter mutation? | Claustres et al (NL#67) |
| | | flanking | | |
| 132C − >G | C to G at 132 | 1 | altered translation | Claustres et al (NL#67) |
| | | | initiation? | |
| P5L | C to T at 146 | 1 | Pro to Leu at codon 5 | Chillón et al. (NL#59) |
| S10R | C to A at 160 | 1 | Ser to Arg at codon 10 | Hughes et al. (NL#65) |
| S13F | C to T at 170 | 1 | Ser to Phe at 13 | Cao et al. (NL#69) |
| 185 + 1G − >T | G to T at 185 + 1 | intron 1 | mRNA splicing defect | Férec 1998* |
| 185 + 4A − >T | A to T at 185 + 4 | intron 1 | mRNA splicing defect? | Culard et al. 1994 |
| | | | (CBAVD) | |
| 186 − 13C − >G | C to G at 186 − 13 | intron 1 | mRNA splicing defect? | Férec et al. (NL#50) |
| W19C | G to T at 189 | 2 | Trp to Cys at 19 | Macek et al. (NL#62) |
| G27E | G to A at 212 | 2 | Gly to Glu at 27 | Bienvenu et al. 1994a |
| R31C | C to T at 223 | 2 | Arg to Cys at 31 | Costes et al. (NL#56) |
| R31L | G to T at 224 | 2 | Arg to Leu at 31 | Zielenski et al. 1995 |
| 232del18 | Deletion of 18 bp from 232 | 2 | Deletion of 6 aa from | Faucz et al. (NL#69) |
| | | | Leu34 to Gln39 | |
| S42F | C to T at 257 | 2 | Ser to Phe at 42 | Férec et al. 1995 |
| D44G | A to G at 263 | 2 | Asp to Gly at 44 | Fanen et al. 1992 |
| A46D | C to A at 269 | 2 | Ala to Asp at 46 | Andoniadi et al. (NL#64) |
| 279A/G | A to G at 279 | 2 | No change (Leu at 49) | Bienvenu et al. (NL#69) |
| I50T | T to C at 280 | 2 | Ile to Thr at codon 50 | Casals et al. (NL#65) |
| S50P | T to C at 280 | 2 | Ser to Pro at 50 | Casals et al. (NL#65) |
| S50Y | C to A at 281 | 2 | Ser to Tyr at 50 | Zielenski et al. (NL#63) |
| | | | (CBAVD) | |
| 296 + 3insT | insertion of T after 296 + 3 | intron 2 | mRNA splicing defect? | Casals et al. 1998* |
| 296 + 1G − >T | G to T at 296 + 1 | intron 2 | missense; mRNA | Walker et al. 2000* |
| | | | splicing defect? | |
| 296 + 1G − >C | G to C at 296 + 1 | intron 2 | mRNA splicing defect | Tzetis et al. (NL#64) |
| 296 + 2T − >C | T to C at 296 + 2 | intron 2 | mRNA splicing defect | Férec et al. (NL#63) |
| 296 + 9A − >T | A to T at 296 + 9 | intron 2 | mRNA splicing defect? | Zielenski et al. (NL#68) |
| 296 + 12T − >C | T to C at 296 + 12 | intron 2 | mRNA splicing defect? | Cuppens et al. (NL#53) |
| 297 − 28insA | insertion of A after 297 − 28 | intron 2 | mRNA splicing defect? | Scheffer & Dijkstra |
| | | | | (NL#60) |
| 297 − 3C − >A | C to A at 297 − 3 | intron 2 | mRNA splicing defect? | Zielenski et al. (NL#70) |
| 297 − 3C − >T | C to T at 297 − 3 | intron 2 | mRNA splicing defect? | Bienvenu et al. (NL#55) |
| 297 − 2A − >G | A to G at 297 − 2 | intron 2 | mRNA splicing defect | Schwarz et al (NL#67) |
| 297 − 10T − >G | C to G at 297 − 10 | intron 2 | splice mutation? | Zielenski et al. 1999* |
| 297 − 12insA | insertion of A at 297 − 12 | intron 3 | splice mutation? | Girodon et al. 1999* |
| E56K | G to A at 298 | 3 | Glu to Lys at 56 | Dörk et al. (NL#69) |
| W57G | T to G at 301 | 3 | Trp to Gly at 57 | Ferrari et al. (NL#47) |
| W57R | T to C at 301 | 3 | Trp to Arg at 57 | Malone et al. (NL#69) |
| D58N | G to A at 304 | 3 | Asp to Asn at 58 | Dörk et al. (NL#69) |
| D58G | A to G at 305 | 3 | Asp to Gly at 58 | Claustres et al. 2000* |
| E60K | G to A at 310 | 3 | Glu to Lys at 60 | Claustres et al. 2000* |
| E60L | G to A at 310 | 3 | Glu to Leu at 60 | Casals et al. 2000* |
| N66S | A to G at 328 | 3 | Asn to Ser at 66 | Cashman et al. (NL#55) |
| P67L | C to T at 332 | 3 | Pro to Leu at 67 | Hamosh et al. (NL#54) |
| K68E | A to G at 334 | 3 | Lys to Glu at 68 | Kilinc et al. (NL#70) |
| K68N | A to T at 336 | 3 | Lys to Asn at 68 | Dörk & Tümmler |
| | | | | (NL#48) |
| A72T | G to A at 346 | 3 | Ala to Thr at 72 | Pacheco et al. 1999* |
| A72D | C to A at 347 | 3 | Ala to Asp at 72 | Le Gall et al. (NL#68) |
| R74W | C to T at 352 | 3 | Arg to Trp at 74 | Claustres et al. 1993 |
| R74Q | G to A at 353 | 3 | Arg to Gln at 74 | Malone et al. 2000* |
| R75L | G to T at 356 | 3 | Arg to Leu at 75 | Costes et al. (NL#55) |
| W79R | T to C at 367 | 3 | Trp to Arg at 79 | Macek et al. (NL#56) |
| G85E | G to A at 386 | 3 | Gly to Glu at 85 | Zielenski et al. 1991b |
| G85V | G to T at 386 | 3 | Gly to Val at 85 | Casals et al. (NL#67) |
| F87L | T to C at 391 | 3 | Phe to Leu at 87 | Bienvenu et al. 1994c |
| L88S | T to C at 395 | 3 | Leu to Ser at 88 | Malone et al. (NL#51) |
| Y89C | A to G at 398 | 3 | Tyr to Cys at 89 | Seia et al. 1999* |
| L90S | T to C at 401 | 3 | Leu to Ser at 90 | Férec 1998* |
| G91R | G to A at 403 | 3 | Gly to Arg at 91 | Guillermit et al. 1993 |
| 405 + 1G − >A | G to A at 405 + 1 | intron 3 | mRNA splicing defect | Dörk et al. 1993e |
| 405 + 3A − >C | A to C at 405 + 3 | intron 3 | mRNA splicing defect? | Hamosh et al. (NL#54) |
| 405 + 4A − >G | A to G at 405 + 4 | intron 3 | mRNA splicing defect? | Ghanem et al. 1994 |
| 406 − 10C − >G | C to G at 406 − 10 | intron 3 | mRNA splicing defect? | Greil et al. (NL#55) |
| 406 − 6T − >C | T to C at 406 − 6 | intron 3 | mRNA splicing defect? | Claustres et al. 1993 |
| 406 − 3T − >C | T to C at 406 − 3 | intron 3 | mRNA splicing defect? | Kilinc et al. (NL#70) |
| 406 − 2A − >G | A to G at 406 − 2 | intron 3 | mRNA splicing defect | Dörk et al. (NL#69) |
| 406 − 2A − >C | A to C at 406 − 2 | intron 3 | mRNA splicing defect | Costes et al. (NL#60) |
| 406 − 1G − >C | G to C at 406 − 1 | intron 3 | mRNA splicing defect | Bonizzato et al. 1992 |
| 406 − 1G − >A | G to A at 406 − 1 | intron 3 | mRNA splicing defect | Wang et al. 1998* |
| 406 − 1G − >T | G to T at 406 − 1 | intron 3 | mRNA splicing defect | Bienvenu et al. (NL#55) |
| E92K | G to A at 406 | 4 | Glu to Lys at 92 | Nunes et al. 1993 |
| A96E | C to A at 419 | 4 | Ala to Glu at 96 | Férec 1998* |
| Q98R | A to G at 425 | 4 | Gln to Arg at 98 | Romey et al. 1995 |
| P99L | C to T at 428 | 4 | Pro to Leu at 99 | Schwartz & |
| | | | | Holmberg (NL#50) |
| I105N | T to A at 446 | 4 | Ile to Asn at 105 | Claustres et al. 2000* |
| S108F | C to T at 455 | 4 | Ser to Phe at 108 | Seydewitz et al. 1995 |
| Y109N | T to A at 457 | 4 | Tyr to Asn at 109 | Schaedel et al. 1998* |
| Y109C | A to G at 458 | 4 | Tyr to Cys at 109 | Schaedel et al. 1994 |
| D110H | G to C at 460 | 4 | Asp to His at 110 | Dean et al. 1990 |
| D110Y | G to T at 460 | 4 | Asp to Tyr at 110 | Casals et al. 2000* |
| D110E | C to A at 462 | 4 | Asp to Glu at 110 | Seia et al. 1999* |
| P111A | C to G at 463 | 4 | Pro to Ala at 111 | Férec et al. (NL#69) |
| P111L | C to T at 464 | 4 | Pro to Leu at 111 | Claustres et al. (NL#62) |
| delta E115 | 3 bp deletion of 475-477 | 4 | deletion of Glu at 115 | Chillón et al. 1995 |
| | | | | (NL#61) |
| E116Q | G to C at 478 | 4 | Glu to Gln at 116 | Walker et al. 2000* |
| E116K | G to A at 478 | 4 | Glu to Lys at 116 | Costes et al. (NL#60) |
| R117C | C to T at 481 | 4 | Arg to Cys at 117 | Dörk et al. 1994b |
| R117H | G to A at 482 | 4 | Arg to His at 117 | Dean et al. 1990 |
| R117P | G to C at 482 | 4 | Arg to Pro at 117 | Feldmann et a. (NL#64) |
| R117L | G to T at 482 | 4 | Arg to Leu at 117 | Férec et al. 1995 |
| A120T | G to A at 490 | 4 | Ala to Thr at 120 | Chillón et al. 1994 |
| I125T | T to C at 506 | 4 | Ile to Thr at 125 | Mittre (NL#70) |
| G126D | G to A at 509 | 4 | Gly to Asp at 126 | Wagner et al 1994 |
| L137R | T to G at 542 | 4 | Leu to Arg at 137 | Chevalier-Porst & Bozon |
| | | | | (NL#70) |
| L137H | T to A at 542 | 4 | Leu to His at 137 | Wallace (NL#69) |
| L138ins | insertion of CTA, TAC or ACT | 4 | insertion of leucine at | Dörk et al. (NL#69) |
| | at nucleotide 544, 545 or 546 | | 138 | |
| H139R | A to G at 548 | 4 | His to Arg at 139 | Férec et al. 1995 |
| P140S | C to T at 550 | 4 | Pro to Ser at 140 | Férec et al. (NL#61) |
| P140L | C to T at 551 | 4 | Pro to Leu at 140 | Tzetis et al. (NL#70) |
| A141D | C to A at 554 | 4 | Ala to Asp at 141 | Gouya et al. (NL#65) |
| H146R | A to G at 569 | 4 | His to Arg at 146 | Bienvenu et al. (NL#68) |
| | | | (CBAVD) | |
| I148T | T to C at 575 | 4 | Ile to Thr at 148 | Bozon et al. 1994 |
| I148N | T to A at 575 | 4 | Ile to Asn at 148 | Casals et al. (NL#69) |
| G149R | G to A at 577 | 4 | Gly to Arg at 149 | Mercier et al. 1995 |
| M152V | A to G at 586 | 4 | Met to Val at 152 | Edkins & Creegan |
| | | | (mutation?) | (NL#54) |
| M152R | T to G at 587 | 4 | Met to Arg at 152 | Yoshimura 1998* |
| 591del18 | deletion of 18 bp from 591 | 4 | deletion of 6 a.a. from | Varon & Reis (NL#64) |
| A155P | G to C at 595 | 4 | Ala to Pro at 155 | Zielenski et al. (NL#70) |
| S158R | A to C at 604 | 4 | Ser to Arg at 158 | Girodon et al. 1999* |
| Y161N | T to A at 613 | 4 | Tyr to Asn at 161 | Claustres et al. 2000* |
| Y161D | T to G at 613 | 4 | Tyr to Asp at 161 | Zielenski et al. 1999* |
| Y161S | A to C at 614 (together with | 4 | Tyr to Ser at 161 | Andrew et al. 1999* |
| | 612T/A) | | | |
| K162E | A to G at 616 | 4 | Lys to Glu at 162 | Tzetis et al. (NL#70) |
| 621G − >A | G to A at 621 | 4 | mRNA splicing defect | Mackova et al. (NL#64) |
| 621 + 1G − >T | G to T at 621 + 1 | intron 4 | mRNA splicing defect | Zielenski et al. 1991b |
| 621 + 2T − >C | T to C at 621 + 2 | intron 4 | mRNA splicing defect | Schwarz et al. (NL#66) |
| 621 + 2T − >G | T to G at 621 + 2 | intron 4 | mRNA splicing defect | Claustres et al. 1993 |
| 621 + 3A − >G | A to G at 621 + 3 | intron 4 | mRNA splicing defect | Tzetis et al. (NL#70) |
| 622 − 2A − >C | A to C at 622 − 2 | intron 4 | mRNA splicing defect | Cuppens et al. 1993 |
| 622 − 1G − >A | G to A at 622 − 1 | intron 4 | mRNA splicing defect | Zielenski et al. (NL#66) |
| L165S | T to C at 626 | 5 | Leu to Ser at 165 | Férec et al. (NL#51) |
| K166Q | A to G at 628 | 5 | Lys to Gln at 166 | Macek et al. |
| | | | | (NL#62; #66) |
| R170C | C to T at 640 | 5 | Arg to Cys at 170 | Férec et al. (NL#62) |
| R170G | C to G at 640 | 5 | Arg to Gly at 170 | Claustres et al. (NL#49) |
| R170H | G to A at 641 | 5 | Arg to His at 170 | Brownsell et al. 2001* |
| I175V | A to G at 655 | 5 | Ile to Val at 175 | Romey et al. 1994a |
| I177T | T to C at 662 | 5 | Ile to Thr at 177 | Bienvenu et al. (NL#68) |
| G178R | G to A at 664 | 5 | Gly to Arg at 178 | Zielenski et al. 1991b |
| Q179K | C to A at 667 | 5 | Gln to Lys at 179 | Zhang & Wong 2000* |
| N186K | C to A at 690 | 5 | Asn to Lys at 186 | Claustres & Carles |
| | | | | (NL#70) |
| N187K | C to A at 693 | 5 | Asn to Lys at 187 | Arduino et al. 1998* |
| D192N | G to A at 706 | 5 | Asp to Asn at 192 | Costes et al. (NL#62) |
| delta D192 | deletion of TGA or GAT from | 5 | deletion of Asp at 192 | Feldmann et al. (NL#66) |
| | 706 or 707 | | | |
| D192G | A to G at 707 | 5 | Asp to Gly at 192 | Audrézet et al. 1994 |
| E193K | G to A at 709 | 5 | Glu to Lys at 193 | Ferrari et al. (NL#62); et |
| | | | | al. Mercier et al. 1995 |
| 711 + 1G − >T | G to T at 711 + 1 | intron 5 | mRNA splicing defect | Zielenski et al. 1991b |
| 711 + 3A − >C | A to C at 711 + 3 | intron 5 | mRNA splicing defect | Macek MJr et al. |
| | | | | (NL#61) |
| 711 + 3A − >G | A to G at 711 + 3 | intron 5 | mRNA splicing defect | Petreska et al. 1994 |
| 711 + 3A − >T | A to T at 711 + 3 | intron 5 | mRNA splicing defect? | Casasl et al. (NL#67) |
| 711 + 5G − >A | G to A at 711 + 5 | intron 5 | mRNA splicing defect | Bisceglia et al. 1994 |
| 711 + 34A − >G | A to G at 711 + 34 | intron 5 | mRNA splicing defect? | Tzetis et al. (NL#68) |
| 712 − 1G − >T | G to T at 712 − 1 | intron 5 | mRNA splicing defect | Chillón et al. (NL#59) |
| G194V | G to T at 713 | 6a | Gly to Val at 194 | Férec 1998* |
| A198P | G to C at 724 | 6a | Ala to Pro at 198 | Walker et al. 1999* |
| H199Y | C to T at 727 | 6a | His to Tyr at 199 | Dörk & Tümmler |
| | | | | (NL#45) |
| H199Q | T to G at 729 | 6a | His to Gln at 199 | Dean et al. (NL#28) |
| V201M | G to A at 733 | 6a | Val to Met al 201 | Férec 1998* |
| P205S | C to T at 745 | 6a | Pro to Ser at 205 | Chillón et al. 1993b |
| L206W | T to G at 749 | 6a | Leu to Trp at 206 | Claustres et al. 1993 |
| L206F | G to T at 750 | 6a | Leu to Phe at 206 | Férec et al. (NL#69) |
| A209S | G to T at 757 | 6a | Ala to Ser at 209 | Férec 1998* |
| E217G | A to G at 782 | 6a | Glu to Gly at 217 | Zielenski et al. (NL#70) |
| Q220R | A to G at 791 | 6a | Gln to Arg at 220 | Férec 1998* |
| C225R | T to C at 805 | 6a | Cys to Arg at 225 | Fanen et al. 1992 |
| L227R | T to G at 812 | 6a | Leu to Arg at 227 | Ghanem et al. (NL#59) |
| V232D | T to A at 827 | 6a | Val to Asp at 232 | Costes et al. (NL#60) |
| | | | (CBAVD) | |
| Q237E | C to G at 841 | 6a | Gln to Glu at 237 | Costes et al. (NL#62) |
| G239R | G to A at 847 | 6a | Gly to Arg at 239 | Zielenski et al. (NL#60) |
| G241R | G to A at 852 | 6a | Gly to Arg at 241 | Férec et al. (NL#69) |
| M243L | A to C at 859 | 6a | Met to Leu at 243 (ATG | Yoshimura 1999* |
| | | | to CTG) | |
| M244K | T to A at 863 | 6a | Met to Lys at 244 | Claustres et al. (NL#64) |
| R248T | G to C at 875 | 6a | Arg to Thr at 248 | Scheffer et al. (NL#70) |
| | | | (CBAVD) | |
| 875 + 1G − >C | G to C at 875 + 1 | intron | mRNA splicing defect | Zielenski et al. (NL#58) |
| | | 6a | | |
| 875 + 1G − >A | G to A at 875 + 1 | intron | mRNA splicing defect | Duarte et al. (NL#63) |
| | | 6a | | |
| 876 − 14del12 | deletion of 12 bp from 876 − 14 | intron | mRNA splicing defect? | Audrézet et al. 1993a |
| | | 6a | | |
| 876 − 10del8 | deletion of 8 bp from 876 − 10 | intron | mRNA splicing defect? | Costes et al. (NL#46, 47) |
| | | 6a | | |
| 876 − 3C − >T | C to T at 876 − 3 | intron | splicing mutation? | Chevalier-Porst & Bozon |
| | | 6a | | 1999* |
| R258G | G to A at 904 | 6b | Arg to Gly at 258 | Mercier et al. 1995 |
| V920L | G to T at 289 | 15 | Val to Leu at 920 | Girodon et al. 1999* |
| M265R | T to G at 926 | 6b | Met to Arg at 265 | Schwarz et al. (NL#65) |
| E278del | deletion of AAG from 965 | 6b | deletion of Glu at 278 | Casals et al. (NL#70) |
| N287Y | A to T at 991 | 6b | Asn to Tyr at 287 | Shrimpton & Borowitz |
| | | | | (NL#69) |
| 994del9 | deletion of TTAAGACAG | 6b | mRNA splicing defect | Zielenski et al. (NL#70) |
| | from 994 | | | |
| 1002 − 3T − >G | T to G at 1002 − 3 | intron | mRNA splicing defect | Mackova et al. (NL#64) |
| | | 6b | | |
| E292K | G to A at 1006 | 7 | Glu to Lys at 292 | Bienvenu et al. (NL#68) |
| R297W | C to T at 1021 | 7 | Arg to Trp at 297 | Dörk et al. (NL#69) |
| R297Q | G to A at 1022 | 7 | Arg to Gln at 297 | Graham et al. 1991 |
| A299T | G to A at 1027 | 7 | Ala to Thr at 299 | Férec 1999* |
| Y301C | A to G at 1034 | 7 | Tyr to Cys at 301 | Constantinou-Deltas |
| | | | | (NL#58) |
| S307N | G to A at 1052 | 7 | Ser to Asn at 307 | Onay & Kirdar (NL#70) |
| A309D | C to A at 1058 | 7 | Ala to Asp at 309 | Ferrari et al. (NL#64) |
| A309G | C to G at 1058 | 7 | Ala to Gly at 309 | Bienvenu et al. (NL#68) |
| delta F311 | deletion of 3 bp between 1059 | 7 | deletion of Phe310, 311 | Meitinger et al. 1993 |
| | and 1069 | | or 312 | |
| F311L | C to G at 1065 | 7 | Phe to Leu at 311 | Férec et al. 1992 |
| G314R | G to C at 1072 | 7 | Gly to Arg at 314 | Nasr et al. (NL#56) |
| G314V | G to T at 1073 | 7 | Gly to Val at 324 | Chevalier-Porst & Bozon |
| | | | | (NL#70) |
| G314E | G to A at 1073 | 7 | Gly to Glu at 314 | Golla et al. 1994 |
| F316L | T to G at 1077 | 7 | Phe to Leu at 316 | Férec 2000* |
| V317A | T to C at 1082 | 7 | Val to Ala at 317 | Férec et al. (NL#55) |
| L320V | T to G at 1090 | 7 | Leu to Val at 320 CAVD | Bienvenu et al (NL#67) |
| L320F | A to T at 1092 | 7 | Leu to Phe at 320 | Macek et al. (NL#64) |
| V322A | T to C at 1097 | 7 | Val to Ala at 322 | Férec et al. (NL#63) |
| | | | (mutation?) | |
| L327R | T to G at 1112 | 7 | Leu to Arg at 327 | Ravnik-Glavac et al. |
| | | | | (NL#53) |
| R334W | C to T at 1132 | 7 | Arg to Trp at 334 | Estivill et al. 1991 |
| R334L | G to T at 1133 | 7 | Arg to Leu at 334 | Dörk et al. (NL#69) |
| R334Q | G to A at 1133 | 7 | Arg to Gln at 334 | Férec et al. (NL#65) |
| I336K | T to A at 1139 | 7 | Ile to Lys at 336 | Cuppens et al. 1993 |
| T338I | C to T at 1145 | 7 | Thr to Ile at 338 | Saba et al. 1993 |
| E474K | G to A at 1152 | 10 | Glu to Lys at 474 | Girodon et al. 1999* |
| L346P | T to C at 1169 | 7 | Leu to Pro at 346 | Constantinou (NL#58) |
| R347C | C to T at 1171 | 7 | Arg to Cys at 347 | Férec et al. (NL#56) |
| R347H | G to A at 1172 | 7 | Arg to His at 347 | Cremonesi et al., 1992 |
| R347P | G to C at 1172 | 7 | Arg to Pro at 347 | Dean et al. (NL#6) |
| R347L | G to T at 1172 | 7 | Arg to Leu at 347 | Audrézet et al. 1993a |
| M348K | T to A at 1175 | 7 | Met to Lys at 348 | Audrézet et al. 1993b |
| A349V | C to T at 1178 | 7 | Ala to Val at 349 | Audrézet et al. 1993a |
| R352W | C to T at 1186 | 7 | Arg to Trp at 352 | Byrne et al. (NL#69) |
| R352Q | G to A at 1187 | 7 | Arg to Gln at 352 | Cremonesi et al. 1992 |
| Q353H | A to C at 1191 | 7 | Gln to His at 353 | Férec et al. (NL#65) |
| Q359K/T360K | C to A at 1207 and C to A at | 7 | Glu to Lys at 359 and Thr | Shoshani et al. 1992 |
| | 1211 | | to Lys at 360 | |
| Q359R | A to G at 1208 | 7 | Gln to Arg at 359 | Férec 1999* |
| W361R(T − >C) | T to C at 1213 | 7 | Trp to Arg at 361 | Bienvenu et al. (NL#56) |
| W361R(T − >A) | T to A at 1213 | 7 | Trp to Arg at 361 | Telleria & Alonso 1998* |
| S364P | T to C at 1222 | 7 | Ser to Pro at 364 | Hamosh et al. (NL#54) |
| L365P | T to C at 1226 | 7 | Leu to Pro at 365 | Casals et al. 2000* |
| 1243ins6 | insertion of ACAAAA after | 7 | insertion of Asp and Lys | Shackleton et al (NL#67) |
| | 1243 | | after Lys370 | |
| 1248 + 1G − >A | G to A at 1248 + 1 | intron 7 | mRNA splicing defect | Schwarz et al. (NL#58) |
| 1249 − 29delAT | deletion of AT from 1249 − 29 | intron 7 | mRNA splicing defect? | Zielenski et al. (NL#69) |
| 1249 − 27delTA | deletion of TA at 1249 − 27 | intron 7 | mRNA splicing defect? | Egan et al. (NL#70) |
| 1249 − 5A − >G | A to G at 1249 | intron 7 | mRNA splicing defect? | Bienvenu et al. (NL#62) |
| L375F | A to C at 1257 | 8 | Leu to Phe at 375 | Jézéquel (NL#65) |
| | | | (CUAVD) | |
| E379X | G to T at 1267 | 8 | Glu to Stp at 379 | Glaeser & Mehnert |
| | | | | 2000* |
| L383S | T to C at 1280 | 8 | Leu to Ser at 383 | Casals et al. (NL#69) |
| T360R | C to G at ? | 7 | Thr to Arg at 360 | Férec 1998* |
| V392A | T to C at 1307 | 8 | Val to Ala at 392 CAVD | Bienvenu et al (NL#67, |
| | | | | NL#68) |
| V392G | T to G at 1307 | 8 | Val to Gly at 392 | Zielenski et al. Larder et |
| | | | | al. (NL#70) |
| M394R | T to G at 1313 | 8 | Met to Arg at 394 | Férec 1998* |
| A399V | C to T at 1328 | 8 | Ala to Val at 399 | Yoshimura & Azuma |
| | | | | 2000* |
| E403D | G to C at 1341 | 8 | Glu to Asp at 403 | Férec 1999* |
| 1341G − >A | G to A at 1341 | 8 | ? | Telleria & Alonso 1998* |
| 1341G − >A | G to A at 1341 | 8 | | Telleria 1999* |
| 1341 + 1G − >A | G to A at 1341 + 1 | intron 8 | mRNA splicing defect | Dörk et al. (NL#69) |
| 1341 + 18A − >C | A to C at 1341 + 18 | intron 8 | mRNA splicing defect? | Claustres et al. (NL#60) |
| 1342 − 11TTT − >G | TTT to G at 1342 − 11 | intron 8 | mRNA splicing defect? | Dörk & Tümmler |
| | | | | (NL#59) |
| 1342 − 2A − >C | A to C at 1342 − 2 | intron 8 | mRNA splicing defect | Dörk et al. 1993b |
| 1342 − 1G − >C | G to C at 1342 − 1 | intron 8 | mRNA splicing defect | Cutting & Curristin (NL |
| | | | | #30) |
| E407V | A to T at 1352 | 9 | Glu to Val at 407 | Zielenski et al. 1999* |
| N418S | A to G at 1385 | 9 | Asn to Ser at 418 | Sava et al. (NL#64) |
| G424S | G to A at 1402 | 9 | Gly to Ser at 424 | Bienvenu et al. 2000* |
| D443Y | G to T at 1459 | 9 | Asp to Tyr at 443 | Bienvenu et al. (NL#63) |
| I444S | T to G at 1463 | 9 | Ile to Ser at 444 | Zielenski et al. 1999* |
| Q452P | A to C at 1487 | 9 | Gln to Pro at 452 | Claustres et al. (NL#70) |
| delta L453 | deletion of 3 bp between 1488 | 9 | deletion of Leu at 452 or | Dörk et al (NL#67) |
| | and 1494 | | 454 | |
| A455E | C to A at 1496 | 9 | Ala to Glu at 455 | Kerem et al. 1990 |
| V456F | G to T at 1498 | 9 | Val to Phe at 456 | Dörk et al. 1994a |
| G458V | G to T at 1505 | 9 | Gly to Val at 458 | Cuppens et al. 1990 |
| 1524 + 6insC | insertion of C after 1524 + 6, | intron 9 | mRNA splicing defect? | Bienvenu et al. (NL#61) |
| | with G to A at 1524 + 12 | | | |
| 1525 − 1G − >A | G to A at 1525 − 1 | intron 9 | mRNA splicing defect | Dörk et al. 1993a |
| S466L | C to T at 1529 | 10 | Ser to Leu at 466 | Costes et al. (NL#66) |
| | | | (CBAVD) | |
| G480S | G to A at 1570 | 10 | Gly to Ser at 480 | Kawasoe et al. 2001* |
| G480C | G to T at 1570 | 10 | Gly to Cys at 480 | Smit et al 1991 |
| G480D | G to A at 1570 | 10 | Gly to Asp at 480 | Hawworth et al. (NL#66) |
| H484Y | C to T at 1582 | 10 | His to Tyr at 484 | Casals et al. (NL#69) |
| | | | (CBAVD?) | |
| H484R | A to G at 1583 | 10 | His to Arg at 484 | Férec 1998* |
| S485C | A to T at 1585 | 10 | Ser to Cys at 485 | Andrew et al. 1999* |
| C491R | T to C at 1603 | 10 | Cys to Arg at 491 | Chevalier-Porst & Bozon |
| | | | | (NL#70) |
| S492F | C to T at 1607 | 10 | Ser to Phe at 492 | Férec et al. 1992 |
| Q493R | A to G at 1610 | 10 | Gln to Arg at 493 | Savov et al. 1994a |
| P499A | C to G at 1627 | 10 | Pro to Ala at 499 | Arduino et al. (NL#68) |
| | | | (CBAVD) | |
| T501A | A to G at 1633 | 10 | Thr to Ala at 501 | Claustres et al. 1999* |
| I502T | T to C at 1637 | 10 | Ile to Thr at 502 | Chevalier-Porst & Bozon |
| | | | | (NL#70) |
| E504Q | G to C at 1642 | 10 | Glu to Gln at 504 | Baranov (NL#34, #35) |
| I506L | A to C at 1648 | 10 | Ile to Leu at 506 | Zielenski et al. (NL#70) |
| delta 1507 | deletion of 3 bp between 1648 | 10 | deletion of Ile506 or | Kerem et al. 1990; |
| | and 1653 | | Ile507 | Schwarz et al. 1991 |
| I506S | T to G at 1649 | 10 | Ile to Ser at 506 | Deufel et al. 1994 |
| I506T | T to C at 1649 | 10 | Ile to Thr at 506 | Desgeorges et al. 1995 |
| delta F508 | deletion of 3 bp between 1652 | 10 | deletion of Phe at 508 | Rommens et al., Riordan |
| | and 1655 | | | et al., Kerem et al. 1989 |
| F508S | T to C at 1655 | 10 | Phe to Ser at 508 | Férec 1998* |
| D513G | A to G at 1670 | 10 | Asp to Gly at 513 | Bienvenu et al. (NL#70) |
| | | | (CBAVD) | |
| Y517C | A to G at 1682 | 10 | Tyr to Cys at 517 | Arduino et al. (NL#70) |
| V520F | G to T at 1690 | 10 | Val to Phe at 520 | Jones et al. 1992 |
| V520I | G to A at 1690 | 10 | Val to Ile at 520 | Malone et al. (NL#60) |
| 1706del16 | 16 bp deletion from 1706 | 10 | deletion of spice site | |
| 1706del17 | deletion of 17 bp ftom 1706 | 10 | deletion of splice site | Leoni et al. 1993 |
| E527Q | G to C at 1711 | 10 | Glu to Gln at 527 | Byrne et al. (NL#70) |
| E527G | A to G at 1712 | 10 | Glu to Gly at 527 | Benetazzo et al. (NL#70) |
| 1716 − 1G − >A | G to A at 1716 − 1 | intron | mRNA splicing defect | Jordanova et al. (NL#69) |
| | | 10 | | |
| E528D | G to T at 1716 | 10 | Glu to Asp at 528 (splice | Girodon et al. 1999* |
| | | | mutation?) | |
| 1716 + 2T − >C | T to C at 1716 + 2 | intron | mRNA splicing defect | Claustres et al. (NL#68) |
| | | 10 | | |
| 1717 − 8G − >A | G to A at 1717 − 8 | intron | mRNA splicing defect? | Savov et al. 1994a |
| | | 10 | | |
| 1717 − 3T − >G | T to G at 1717 − 3 | intron | mRNA splicing defect? | Férec et al. (NL#68) |
| | | 10 | | |
| 1717 − 2A − >G | A to G at 1717 − 2 | intron | mRNA splicing defect | Hawworth et al (NL#67) |
| | | 10 | | |
| 1717 − 1G − >A | G to A at 1717 − 1 | intron | mRNA splicing defect | Kerem et al. 1990 |
| | | 10 | | |
| 1717 − 9T − >A | T to A at 1717 − 9 | intron | mRNA splicing | Vouk & Komel 1999* |
| | | 10 | mutation? | |
| D529H | G to C at 1717 | 11 | Asp to His at 529 | Férec 1998* |
| A534E | C to A at 1733 | 11 | Ala to Glu at 534 | Audrézet et al. 1993a |
| I539T | T to C at 1748 | 11 | Ile to Thr at 539 | Chomel & Kitzis |
| | | | | (NL#66) |
| G544S | G to A at 1762 | 11 | Gly to Ser at 544 | Férec et al. (NL#61) |
| G544V | G to T at 1763 | 11 | Gly to Val at 544 | Claustres et al. (NL#69) |
| | | | (CBAVD) | |
| S549R(A − >C) | A to C at 1777 | 11 | Ser to Arg at 549 | Sangiuolo et al. 1990 |
| S549N | G to A at 1778 | 11 | Ser to Asn at 549 | Cutting et al. 1990a |
| S549I | G to T at 1778 | 11 | Ser to Ile at 549 | Kerem et al. 1990 |
| S549R(T − >G) | T to G at 1779 | 11 | Ser to Arg at 549 | Kerem et al. 1990 |
| G550R | G to A at 1780 | 11 | Gly to Arg at 550 | Férec et al. (NL#66) |
| G551S | G to A at 1783 | 11 | Gly to Ser at 551 | Strong et al. 1991 |
| G551D | G to A at 1784 | 11 | Gly to Asp at 551 | Cutting et al. 1990a |
| Q552K | C to A at 1786 | 11 | Gln to Lys | Faucz et al. (NL#69) |
| R553G | C to G at 1789 | 11 | Arg to Gly at 553 | Férec et al. (NL#59) |
| R553Q | G to A at 1790 | 11 | Arg to Gln at 553 | Dörk et al. 1991b |
| | | | (associated with delta | |
| | | | F508; | |
| R555G | A to G at 1795 | 11 | Arg to Gly at 555 | Zielenski et al 1999* |
| I556V | A to G at 1798 | 11 | Ile to Val at 556 | Ghanem et al. (NL#50) |
| | | | (mutation?) | |
| L558S | T to C at 1805 | 11 | Leu to Ser at 558 | Maggio et al. (NL#31) |
| A559T | G to A at 1807 | 11 | Ala to Thr at 559 | Cutting et al. 1990a |
| A559E | C to A at 1808 | 11 | Ala to Glu at 559 | Girodon et al. 1999* |
| R560K | G to A at 1811 | 11 | Arg to Lys at 560 | Férec et al. 1992 |
| R560T | G to C at 1811 | 11 | Arg to Thr at 560; | Kerem et al. 1990 |
| | | | mRNA splicing defect? | |
| 1811 + 1G − >C | G to C at 1811 + 1 | intron | mRNA splicing defect | Petreska et al. (NL#50) |
| | | 11 | | |
| 1811 + 1.6kbA − >G | A to G at 1811 + 1.2 kb | intron | creation of splice donor | Chillón et al. 1995 |
| | | 11 | site | |
| 1811 + 18G − >A | G to A at 1811 + 18 | intron | mRNA splicing defect? | Teng et al. (NL#65) |
| | | 11 | | |
| 1812 − 1G − >A | G to A at 1812 − 1 | intron | mRNA splicing defect | Chillón et al. 1994 |
| | | 11 | | |
| R560S | A to C at 1812 | 12 | Arg to Ser at 560 | Costes et al. (NL#54) |
| A561E | C to A at 1814 | 12 | Ala to Glu at 561 | Duarte et al. (NL#55) |
| V562L | G to C at 1816 | 12 | Val to Leu at 562 | Hughes et al. (NL#65) |
| V562I | G to A at 1816 | 12 | Val to Ile at 562 | Feldmann et al (NL#67) |
| Y563D | T to G at 1819 | 12 | Tyr to Asp at 563 | Hamosh et al. (NL#54) |
| Y563N | T to A at 1819 | 12 | Tyr to Asn at 563 | Kerem et al. (NL#13) |
| Y563C | A to G at 1821 | 12 | Tyr to Cys at 563 | Delhaize C (NL#67) |
| L568F | G to T at 1836 | 12 | Leu to Phe at 568 | Dörk et al. (NL#69) |
| | | | (CBAVD?) | |
| Y569D | T to G at 1837 | 12 | Tyr to Asp at 569 | Malone et al. (NL#65) |
| Y569H | T to C at 1837 | 12 | Tyr to His at 569 | Costes et al. (NL#52) |
| Y569C | A to G at 1838 | 12 | Tyr to Cys at 569 | Plaseska et al. (NL#45) |
| LS71S | T to C at 1844 | 12 | Leu to Ser at 571 | Savov et al. (NL#60) |
| D572N | G to A at 1846 | 12 | Asp to Asn at 572 | Férec et al. (NL#59) |
| P574H | C to A at 1853 | 12 | Pro to His at 574 | Kerem et al. 1990 |
| G576A | G to C at 1859 | 12 | Gly to Ala at 576 | Sarginson et al. (NL#69) |
| | | | (CAVD) | |
| Y577F | A to T at 1862 | 12 | Tyr to Phe at 577 | Dörk et al (NL#67) |
| D579Y | G to T at 1867 | 12 | Asp to Tyr at 579 | Harris et al. (NL#63) |
| D579G | A to G at 1868 | 12 | Asp to Gly at 579 | Ferrari et al. (NL#53) |
| D579A | A to C at 1868 | 12 | Asp to Ala at 579 | Pacheco et al. (NL#70) |
| T582I | C to T at 1877 | 12 | Thr to Ile at 582 | Claustres et al (NL#67) |
| T582R | C to G at 1877 | 12 | Thr to Arg at 582 | Casals et al. (NL#55) |
| S589N | G to A at 1898 | 12 | Ser to Asn at 589 | Scheffer et al. (NL#68) |
| | | | (mRNA splicing defect?) | |
| S589I | G to T at 1898 | 12 | Ser to Ile at 589 | Schwarz et al. 1999* |
| | | | (splicing?) | |
| 1898 + 1G − >T | G to T at 1898 + 1 | intron | mRNA splicing defect | Morris (NL#62) |
| | | 12 | | |
| 1898 + 1G − >C | G to C at 1898 + 1 | intron | mRNA splicing defect | Cuppens et al. 1993 |
| | | 12 | | |
| 1898 + 1G − >A | G to A at 1898 + 1 | intron | mRNA splicing defect | Strong et al. 1992 |
| | | 12 | | |
| 1898 + 3A − >C | A to C at 1898 + 3 | intron | mRNA splicing defect? | Mercier et al. 1995 |
| | | 12 | | |
| 1898 + 3A − >G | A to G at 1898 + 3 | intron | mRNA splicing defect? | Ferrari et al. (NL#35) |
| | | 12 | | |
| 1898 + 5G − >T | G to T at 1898 + 5 | intron | mRNA splicing defect | Zielenski et al. 1995 |
| | | 12 | | |
| 1898 + 5G − >A | G to A at 1898 + 5 | intron | mRNA splicing defect | Férec et al. (NL#69) |
| | | 12 | | |
| 1898 + 73T − >G | T to G at 1898 + 73 | intron | mRNA splicing defect? | Smit et al. (NL#37) |
| | | 12 | | |
| R600G | A to G at 1930 | 13 | Arg to Gly at 600 | Bienvenu et al. (NL#69) |
| I601F | A to T at 1933 | 13 | Ile to Phe at 601 | Schwarz et al. (NL#68) |
| V603F | G to T at 1939 | 13 | Val to Phe at 603 | Zielenski et al. (NL#70) |
| T604I | C to T at 1943 | 13 | Thr to Ile at 604 | Girodon et al. 1999* |
| 1949del84 | deletion of 84 bp from 1949 | 13 | deletion of 28 a.a. | Granell et al. 1992 |
| | | | (Met607 to Gln634) | |
| H609R | A to G at 1958 | 13 | His to Arg at 609 | Bienvenu et al. (NL#69) |
| L610S | T to C at 1961 | 13 | Leu to Ser at 610 | Férec et al. (NL#52) |
| A613T | G to A at 1969 | 13 | Ala to Thr at 613 | Liechti-Gallati (NL#68) |
| D614Y | G to T at 1972 | 13 | Asp to Tyr 614 | Girodon et al. 1999* |
| D614G | A to G at 1973 | 13 | Asp to Gly at 614 | Audrézet et al. 1993b |
| I618T | T to C at 1985 | 13 | Ile to Thr at 618 | Macek et al. (NL#62) |
| L619S | T to C at 1988 | 13 | Leu to Ser at 619 | Dörk et al. 1991 |
| H620P | A to C at 1991 | 13 | His to Pro at 620 | Haworth et al. (NL#66) |
| H620Q | T to G at 1992 | 13 | His to Gln at 620 | Dörk and Sturhmann |
| | | | | (NL#68) |
| G622D | G to A at 1997 | 13 | Gly to Asp at 622 | Zielenski et al. (NL#68) |
| | | | (oligospermia) | |
| G628R(G − >A) | G to A at 2014 | 13 | Gly to Arg at 628 | Fanen et al. 1992 |
| G628R(G − >C) | G to C at 2014 | 13 | Gly to Arg at 628 | Cuppens et al. 1993 |
| L633P | T to C at 2030 | 13 | Leu to Pro at 633 | Haworth et al. (NL#62) |
| L636P | T to C at 2039 | 13 | Leu to Pro at 636 | Bombieri et al. (NL#70) |
| D648V | A to T at 2075 | 13 | Asp to Val at 648 | Férec et al. (NL#44) |
| D651N | G to A at 2083 | 13 | Asp to Asn at 651 | Bombieri et al.(NL#70) |
| T665S | A to T at 2125 | 13 | Thr to Ser at 665 | Férec et al. (NL#63) |
| E672del | deletion of 3 bp between 2145- | 13 | deletion of Glu at 672 | Claustres et al. (NL#69) |
| | 2148 | | | |
| K683R | A to G at 2180 | 13 | Lys to Arg at 683 | Chevalier-Porst & Bozon |
| | | | | 2000* |
| F693L(CTT) | T to C at 2209 | 13 | Phe to Leu at 693 | Audrézet et al. 1993b |
| F693L(TTG) | T to G at 2211 | 13 | Phe to Leu at 693 | Meyer et al. 2001* |
| K698R | A to G 2225 | 13 | Lys to Arg at 698 | Férec et al. (NL#69) |
| E725K | G to A at 2305 | 13 | Glu to Lys at 725 | Tzetis et al. (NL#70) |
| P750L | C to T at 2381 | 13 | Pro to Leu at 750 | Chevalier-Porst & Bozon |
| | | | | 2000* |
| V754M | G to A at 2392 | 13 | Val to Met al 754 | Wallace (NL#69) |
| T760M | C to T at 2411 | 13 | Thr to Met al 760 | Zielenski et al. 1999* |
| R766M | G to T at 2429 | 13 | Arg to Met al 766 | Glavac et al. (NL#66) |
| N782K | C to A at 2478 | 13 | Asn to Lys at 782 | Girodon et al. 1999* |
| R792G | C to G at 2506 | 13 | Arg to Gly at 792 | Glavac et al. (NL#66) |
| A800G | C to G at 2531 | 13 | Ala to Gly at 800 | Mercier et al. 1995 |
| E822K | G to A at 2596 | 13 | Glu to Lys at 822 | Mercier et al. 1993a |
| E826K | G to A at 2608 | 13 | Glu to Lys at 826 | Bombieri et al (NL#67) |
| 2622 + 1G − >T | G to T at 2622 + 1 | intron | splice mutation | Girodon et al. 1999* |
| | | 13 | | |
| 2622 + 1G − >A | G to A at 2622 + 1 | intron | mRNA splicing defect | Audrézet et al. 1993a |
| | | 13 | | |
| 2622 + 2del6 | deletion of TAGGTA from | intron | mRNA splicing defect | Zielenski et al. (NL#70) |
| | 2622 + 2 | 13 | | |
| D836Y | G to T at 2638 | 14a | Asp to Tyr at 836 | Ghanem & Goossens |
| | | | | (NL#47) |
| R851L | G to T at 2684 | 14a | Arg to Leu at 851 | Casals et al. (NL#68) |
| C866Y | G to A at 2729 | 14a | Cys to Tyr at 866 | Audrézet et al. (NL#41) |
| L867X | T to A at 2732 | 14a | Leu to Stop at 867 | Haworth et al. (NL#69) |
| 2751G − >A | G to A at 2751 | 14a | mRNA splicing defect? | Wagner et al. (NL#65) |
| 2751 + 2T − >A | T to A at 2751 + 2 | intron | mRNA splicing defect | Antoniadi et al. (NL#68) |
| | | 14a | | |
| 2751 + 3A − >G | A to G at 2751 + 3 | intron | mRNA splicing defect? | Casals et al. (NL#65) |
| | | 14a | (CBAVD) | |
| 2752 − 26A − >G | A to G at 2752 − 26 | intron | mRNA splicing defect? | Tzetis et al. (NL#66) |
| | | 14a | | |
| 2752 − 1G − >T | G to T at 2752 − 1 | intron | mRNA splicing defect | Férec et al. (NL#65) |
| | | 14a | | |
| 2752 − 1G − >C | G to C at 2752 − 1 | intron | splice mutation | Dubourg & Blayau |
| | | 14a | | 1999* |
| T908N | C to A at 2788 | 14b | Thr to Asn at 908 | Férec et al. (NL#69) |
| 2789 + 2insA | insertion of A after 2789 + 2 | intron | mRNA splicing defect? | Dubourg et al. (NL#70) |
| | | 14b | (CAVD) | |
| 2789 + 3delG | deletion of G at 2789 + 3 | intron | mRNA splicing defect | Macek et al. (NL#63) |
| | | 14b | | |
| 2789 + 5G − >A | G to A at 2789 + 5 | intron | mRNA splicing defect | Highsmith et al. 1990 |
| | | 14b | | |
| 2790 − 2A − >G | A to G at 2790 − 2 | intron | mRNA splicing defect | Marigo et al. (NL#61) |
| | | 14b | | |
| 2790 − 1G − >C | G to C at 2790 − 1 | intron | mRNA splicing defect | Schwartz et al. (NL#54) |
| | | 14b | | |
| 2790 − 1G − >T | C to T at 2790 − 1 | intron | mRNA splicing defect | Bienvenu et al. (NL#63) |
| | | 14b | | |
| Q890R | A to G at 2801 | 15 | Gln to Arg at 890 | Casals et al. 1998* |
| D891G | A to G at 2804 | 15 | Asp to Gly at 891 | Kilinc et al. (NL#70) |
| S895T | G to T at 2816 | 15 | Ser to Thr at 895 | Férec 1999* |
| T896I | C to T at 2819 | 15 | Thr to Ile at 896 | Lázaro et al. 2000* |
| N900T | G to A at 2831 | 15 | Asn to Thr at 900 | Férec 1999* |
| 2851A/G | A or G at 2851 | 15 | Ile or Val at 907 | Claustres et al. 2000* |
| S912L | C to T at 2867 | 15 | Ser to Leu at 912 | Ghanem et al. 1994 |
| Y913C | A to G at 2870 | 15 | Tyr to Cys at 913 | Vidaud et al. 1990 |
| Y917D | T to G at 2881 | 15 | Tyr to Asp at 917 | Schwarz et al. (NL#69) |
| Y917C | A to G at 2882 | 15 | Tyr to Cys at 917 | Edkins & Creegan |
| | | | | (NL#60) |
| I918M | T to G at 2886 | 15 | Ile to Met al 918 | Girodon et al. 1999* |
| Y919C | A to G at 2888 | 15 | Tyr to Cys at 919 | Savov et al. 1994a |
| V920M | G to A at 2890 | 15 | Val to Met al 920 | Bienvenu et al. (NL#63) |
| D924N | G to A at 2902 | 15 | Asp to Asn at 924 | Girodon et al. 1999* |
| L927P | T to G at 2912 | 15 | Leu to Pro at 927 | Hermans et al. 1994 |
| F932S | T to C at 2927 | 15 | Phe to Ser at 932 | Férec 1999* |
| R933S | A to T at 2931 | 15 | Arg to Ser at 933 | Dörk et al. (NL#69) |
| | | | (CBAVD) | |
| V938G | T to G at 2945 | 15 | Val to Gly at 938 | Dörk et al. (NL#69) |
| | | | (CAVD) | |
| H939D | C to G at 2947 | 15 | His to Asp at 939 | Férec et al. (NL#54) |
| H939R | A to G at 2948 | 15 | His to Arg at 939 | Férec et al. (NL#69) |
| S945L | C to T at 2966 | 15 | Ser to Leu at 945 | Claustres et al. 1993 |
| K946X | A to T at 2968 | 15 | Lys to Stop at 946 | Haworth et al. (NL#69) |
| H949Y | C to T at 2977 | 15 | His to Tyr at 949 | Ghanem et al. 1994 |
| H949R | A to G at 2978 | 15 | His to Arg at 949 | Férec et al. (NL#65) |
| M952T | T to C at 2987 | 15 | Met to Thr at 952 | Zielenski et al. 1999* |
| M952I | G to C at 2988 | 15 | Met to Ile at 952 | Girodon et al (NL#67) |
| | | | CBAVD mutation? | |
| M961I | G to T at 3015 | 15 | Met to Ile at 961 | Malone et al. 2000* |
| L967S | T to C at 3032 | 15 | Leu to Ser at 967 | Zielenski et al. (NL#70) |
| | | | (oligospermia?) | |
| G970R | G to C at 3040 | 15 | Gly to Arg at 970 | Cuppens et al. 1993 |
| 3040 + 2T − >C | T to C at 3040 + 2 | intron | mRNA splicing defect | Poncin (NL#69) |
| | | 15 | | |
| 3041 − 1G − >A | G to A at 3041 − 1 | intron | mRNA splicing defect | Malone et al (NL#67) |
| | | 15 | | |
| G970D | G to A at 3041 | 16 | Gly to Asp at 970 | Vassilakis et al. (NL#69) |
| L973F | TC to AT at 3048 and 3049 | 16 | Leu to Phe at 973 | Dörk and Sturhmann |
| | | | CBAVD) | (NL#68) |
| L973P | T to C at 3050 | 16 | Leu to Pro at 973 | Férec 1998* |
| S977P | T to C at 3061 | 16 | Ser to Pro at 977 | Dörk et al. (NL#51) |
| S977F | C to T at 3062 | 16 | Ser to Phe at 977 | Férec et al. (NL#69) |
| D979V | A to T at 3068 | 16 | Asp to Val at 979 | Feldmann et al. (NL#68) |
| D979A | A to C at 3068 | 16 | Asp to Ala at 979 | Dörk and Sturhmann |
| | | | (CBAVD?) | (NL#68) |
| I980K | T to A at 3071 | 16 | Ile to Lys at 980 | Bienvenu et al. (NL#62) |
| D985H | G to C at 3085 | 16 | Asp to His at 985 | Claustres & Guittard |
| | | | | (NL#70) |
| D985Y | G to T at 3085 | 16 | Asp to Tyr at 985 | Bienvenu et al. (NL#63) |
| I991V | A to G at 3103 | 16 | Ile to Val at 991 | Bombieri et al. 2000* |
| D993Y | G to T at 3109 | 16 | Asp to Tyr at 993 | Claustres et al (NL#67) |
| F994C | T to G at 3113 | 16 | Phe to Cys at 994 | Claustres et al. (NL#70) |
| 3120G − >A | G to A at 3120 | 16 | mRNA splicing defect | Zielenski et al. 1994 |
| 3120 + 1G − >A | G to A at 3120 + 1 | intron | mRNA splicing defect | Macek et al. (1997) |
| | | 16 | | |
| 3121 − 2A − >T | A to T at 3121 − 2 | intron | mRNA splicing defect | Férec et al. 1995 |
| | | 16 | | |
| 3121 − 2A − >G | A to G at 3121 − 2 | intron | mRNA splicing defect | Macek et al. (NL#60) |
| | | 16 | | |
| 3121 − 1G − >A | G to A at 3121 − 1 | intron | mRNA splicing defect | Feldmann et al (NL#67) |
| | | 16 | | |
| L997F | G to C at 3123 | 17a | Leu to Phe at 997 | Kabra et al. (NL#69) |
| 3131del15 | deletion of 15 bp from 3130, | 17a | deletion of Val at 1001 to | Wallace & Tassabehji |
| | 3131, or 3132 | | Ile at 1005 | (NL#61) |
| I1005R | T to G at 3146 | 17a | Ile to Arg at 1005 | Dörk et al. 1994b |
| A1006E | C to A at 3149 | 17a | Ala to Glu at 1006 | Férec et al. 1995 |
| V1008D | T to A at 3155 | 17a | Val to Asp at 1008 | Casals et al. (NL#70) |
| A1009T | G to A at 3157 | 17a | Ala to Thr at 1009 | Bombieri et al. 2000* |
| P1013L | C to T at 3169 | 17a | Pro to Leu at 1013 | Onay et al. (NL#69) |
| Y1014C | A to G at 3173 | 17a | Tyr to Cys at 1014 | Bozon (NL#70) |
| P1021S | C to T at 3193 | 17a | Pro to Ser at 1021 | Casals et al. (NL#69) |
| | | | (CBAVD) | |
| 3195del6 | deletion of AGTGAT from | 17a | deletion of Val1022 and | Claustres et al. 1994 |
| | 3195 to 3200 | | Ile1023 | |
| 3196del54 | deletion of 54 bp from 3196 | 17a | deletion of 18 aa from | Desgeorges et al. |
| | | | codon 1022 | (NL#65) |
| 3199del6 | deletion of ATAGTG from | 17a | deletion of Ile at 1023 | Bozon (NL#70) |
| | 3199 | | and Val at 1024 | |
| I1027T | T to C at 3212 | 17a | Ile to Thr at 1027 | Andrew et al. 2001* |
| M1028R | T to G at 3215 | 17a | Met to Arg at 1028 | Lázaro et al. 2000* |
| M1028I | G to T at 3216 | 17a | Met to Ile at 1028 | Onay et al (NL#69) |
| Y1032C | A to G at 3227 | 17a | Tyr to Cys at 1032 | Dörk et al. (NL#69) |
| | | | (CBAVD) | |
| I1366T | T to C at 4229 | 22 | Iso to Thr at 1366 | Férec 1999* |
| 3271delGG | deletion of GG at 3271 | 17a | framshift for exon 17b, | Wang 1998* |
| | | | loss of splice site | |
| 3271 + 1G − >A | G to A at 3271 + 1 | intron | mRNA splicing defect | Mercier et al. 1994 |
| | | 17a | | |
| 3271 + 1delGG | deletion of GG at 3271 + 1 | intron | mRNA splicing defect | Wang et al. 1998* |
| | | 17b | | |
| 3272 − 26A − >G | A to G at 3272 − 26 | intron | mRNA splicing defect? | Fanen et al. 1992 |
| | | 17a | | |
| 3272 − 9A − >T | A to T at 3272 − 9 | intron | mRNA splicing defect? | Chomel et al (NL#67) |
| | | 17a | | |
| 3272 − 4A − >G | A to G at 3272 − 4 | intron | mRNA splicing defect? | Kanvakis (NL#63) |
| | | 17a | | |
| 3272 − 1G − >A | G to A at 3272 − 1 | intron | mRNA splicing defect | Mercier et al. 1993b |
| | | 17a | | |
| G1047D | G to A at 3272 | 17b | Gly to Asp at 1047 and | Teng et al. (NL#68) |
| | | | mRNA splicing defect? | |
| | | | (CBAVD?) | |
| F1052V | T to G at 3286 | 17b | Phe to Val at 1052 | Mercier et al. 1993b |
| T1053I | C to T at 3290 | 17b | missense mutation | Bienvenu et al. 1998* |
| T1053I | C to T at 3290 | 17b | Thr to Ile at 1053 | Bienvenu et al. (1998) |
| | | | (CBAVD?) | |
| H1054D | C to G at 3292 | 17b | His to Asp at 1054 | Férec et al. 1993 |
| T1057A | A to G at 3301 | 17b | Thr to Ala at 1057 | Ghanem et al. (NL#68) |
| K1060T | A to C at 3311 | 17b | Lys to Thr at 1060 | Casals et al. 1995 |
| | | | | (NL#61) |
| G1061R | G to C at 3313 | 17b | Gly to Arg at 1061 | Mercier et al. 1993b |
| L1065F | C to T at 3325 | 17b | Leu to Phe at 1065 | Tzetis et al. (NL#70) |
| L1065R | T to G at 3326 | 17b | Leu to Arg at 1065 | Casals et al (NL#67) |
| L1065P | T to C at 3326 | 17b | Leu to Pro at 1065 | Ghanem et al. 1994 |
| R1066S | C to A at 3328 | 17b | Arg to Ser at 1066 | Férec et al. (NL#65) |
| R1066C | C to T at 3328 | 17b | Arg to Cys at 1066 | Fanen et al. 1992 |
| R1066H | G to A at 3329 | 17b | Arg to His at 1066 | Férec et al. 1992 |
| R1066L | G to T at 3329 | 17b | Arg to Leu at 1066 | Mercier et al. 1993b |
| A1067T | G to A at 3331 | 17b | Ala to Thr at 1067 | Férec et al. 1992 |
| A1067D | C to A at 3332 | 17b | Ala to Asp at 1067 | Girodon et al. 1999* |
| G1069R | G to A at 3337 | 17b | Gly to Arg at 1069 | Savov et al. 1994a |
| R1070W | C to T at 3340 | 17b | Arg to Trp at 1070 | Macek et al. (NL#58) |
| R1070Q | G to A at 3341 | 17b | Arg to Gln at 1070 | Mercier et al. 1993b |
| R1070P | 33341 G to C | 17b | Arg to Pro at 1070 | Shrimpton & Borowitz |
| Q1071P | A to C at 3344 | 17b | Gln to Pro at 1071 | Ghanem et al. 1994 |
| Q1071H | G to T at 3345 | 17b | Glu to His at 1071 | Clasutres et al. 2000* |
| P1072L | C to T at 3347 | 17b | Pro to Leu at 1072 | Bombieri et al. (NL#70) |
| F1074L | T to A at 3354 | 17b | Phe to Leu at 1074 | Casals et al. (NL#65) |
| L1077P | T to C at 3362 | 17b | Leu to Pro at 1077 | Bozon et al. 1994 |
| H1085R | A to G at 3386 | 17b | His to Arg at 1085 | Mercier et al. 1993b |
| T1086I | C to T at 3389 | 17b | Thr to Ile at 1086 | Bienvenu et al (NL#67) |
| N1088D | A to G at 3394 | 17b | Asn to Asp at 1088 | Zielenski et al. (NL#70) |
| Y1082H | T to C at 3406 | 17b | Tyr to His at 1082 | Egan et al. (NL#69) |
| L1093P | T to C at 3410 | 17b | Leu to Pro at 1093 | Wine et al. (NL#69) |
| L1096R | T to G at 3419 | 17b | Leu to Arg at 1096 | Claustres & Guittard |
| | | | | 1998* |
| W1098R | T to C at 3424 | 17b | Trp to Arg at 1098 | Zielenski et al. 1995 |
| Q1100P | A to C at 3431 | 17b | Gln to Pro at 1100 | Nunes et al. (NL#55) |
| M1101R | T to G at 3434 | 17b | Met to Arg at 1101 | Mercier et al. 1993b |
| M1101K | T to A at 3434 | 17b | Met to Lys at 1101 | Zielenski et al. 1993 |
| S1118F | C to T at 3485 | 17b | Ser to Phe at 1118 | Férec 1998* |
| S1118C | C to G at 2485 | 17b | Ser to Cys at 1118 | Zielenski et al. 1999* |
| G1123R | G to C at 3499 | 17b | Gly to Arg at 1123 | Wallace & Tassabehji |
| | | | mRNA splicing defect? | (NL#60) |
| 3499 + 2T − >C | T to C at 3499 + 2 | intron | mRNA splicing defect | Creegan & Edkins |
| | | 17b | | (NL#64) |
| 3499 + 3A − >G | A to G at 3499 + 3 | intron | mRNA splicing defect? | Haworth et al. (NL#68) |
| | | 17b | | |
| 3499 + 6A − >G | A to G at 3499 + 6 | intron | mRNA splicing defect? | Férec et al. (NL#65) |
| | | 17b | | |
| 3500 − 2A − >G | A to G at 3500 − 2 | intron | mRNA splicing defect | Vidaud et al. (NL#70) |
| | | 17b | | |
| E1123del | Deletion of AAG at 3504- | 18 | deletion of Glu at 1123 | Ellis (NL#70) |
| | 3506 | | | |
| G1127E | G to A at 3512 | 18 | Gly to Glu at 1127 | Bienvenu et al. (NL#63) |
| 3523A − >G | A to G at 3523 | 18 | Ile to Val at 1131 | Giorgi et al. 1999* |
| A1136T | G to A at 3538 | 18 | Ala to Thr at 1136 | Férec 2000* |
| M1137V | A to G at 3541 | 18 | Met to Val at 1137 | Zielenski et al. (NL#59) |
| M1137R | T to G at 3542 | 18 | Met to Arg at 1137 | Duarte et al. (NL#65) |
| I1139V | A to G at 3547 | 18 | Ile to Val at 1139 | Teng et al. 1994 |
| delta M1140 | deletion of 3 bp between 3550 | 18 | deletion of Met al 1140 | Férec et al. (NL#64) |
| | and 3553 | | | |
| M1140K | T to A at 3551 | 18 | Met to Lys at 1140 | Férec 1998* |
| T1142I | C to T at 3557 | 18 | Thr to Ile at 421 | Lázaro et al. 2000* |
| V1147I | G to A at 3571 | 18 | Val to Ile at 1147 | Kilinc et al. (NL#70) |
| N1148K | C to A at 3576 | 18 | Asn to Lys at 1148 | Casals et al. 2000* |
| D1152H | G to C at 3586 | 18 | Asp to His at 1152 | Highsmith et al. (NL#49) |
| V1153E | T to A at 3590 | 18 | Val to Glu at 1153 | Dörk et al. (NL#68) |
| | | | (CBAVD) | |
| D1154G | A to G at 3593 | 18 | Asp to Gly at 1154 | Costes et al. (NL#64) |
| | | | (CBAVD) | |
| 3600G − >A | G to A at 3600 | 18 | mRNA splicing defect | Zielenski et al. 1994 |
| 3600 + 2insT | insertion of T after 3600 + 2 | intron | mRNA splicing defect? | Zielenski et al. (NL#70) |
| | | 18 | | |
| 3600 + 5G − >A | G to A at 3600 + 5 | intron | mRNA splicing defect? | Bienvenu et al. (NL#66) |
| | | 18 | | |
| 3601 − 20T − >C | T to C at 3601 − 20 | intron | mRNA splicing mutant? | Kabra et al. (NL#69) |
| | | 18 | | |
| 3601 − 17T − >C | T to C at 3601 − 17 | intron | mRNA splicing defect? | Audrézet et al. 1993a |
| | | 18 | | |
| 3601 − 2A − >G | A to G at 3601 − 2 | intron | mRNA splicing defect | Dörk et al. 1993a |
| | | 18 | | |
| S1159P | T to C at 3607 | 19 | Ser to Pro at 115p | Macek et al. (NL#55) |
| S1159F | C to T at 3608 | 19 | Ser to Phe at 1159 | Férec 1999* |
| D1168G | A to G at 3635 | 19 | Asp to Gly at 1168 | Macek et al. (NL#58) |
| K1177R | A to G at 3662 | 19 | Lys to Arg at 1177 | Baralle et al. (NL#61) |
| 3696G/A | G to A at 3696 | 18 | No change to Ser at 1188 | Malone et al. 1999* |
| V1190P | T to A at 3701 | 19 | Val to Pro at 1190 | Glavac et al. (NL#64) |
| 3750delAG | deletion of AG from 3750 | 19 | frameshift | Mercier et al. 1993a |
| 3755delG | deletion of G between 3751 and | 19 | frameshift | Claustres et al. (NL#70) |
| | 3755 | | | |
| M1210I | G to A at 3762 | 19 | Met to Ile at 1210 | Nukiwa & Seyama |
| | | | | (NL#55) |
| V1212I | G to A at 3766 | 19 | Val to Ile at 1212 | Macek et al. (NL#55) |
| L1227S | T to C at 3812 | 19 | Leu to Ser at 1227 | Dubourg & David |
| | | | | (NL#70) |
| E1228G | A to G at 3815 | 19 | Glu to Gly at 1228 | Kilinc et al. 2000* |
| I1230T | T to C at 3821 | 19 | Ile to Thr at 1230 | Claustres & Maugard |
| | | | | (NL#69) |
| I1234V | A to G at 3832 | 19 | Ile to Val at 1234 | Claustres et al. 1992b |
| S1235R | T to G at 3837 | 19 | Ser to Arg at 1235 | Cuppens et al. 1993 |
| G1237S | G to A at 3841 | 19 | Gly to Ser at 1237 | Casals et al. 2000* |
| Q1238R | A to G at 3845 | 19 | Gln to Arg at 1238 | Férec C et al. (NL#58) |
| 3849G − >A | G to A at 3849 | 19 | mRNA splicing defect? | Cutting et al. 1992 |
| 3849 + 1G − >A | G to A at 3849 + 1 | intron | mRNA splicing defect | Greil et al. 1993 |
| | | 19 | | |
| 3849 + 4A − >G | A to G at 3849 + 4 | intron | mRNA splicing defect? | Ronchetto et al. 1992 |
| | | 19 | | |
| 3849 + 10kbC − >T | C to T in a 6.2 kb EcoRI | intron | creation of splice | Highsmith et al. 1994 |
| | fragment 10 kb from 19 | 19 | acceptor site | |
| 3849 + 5G − >A | G to A at 3849 + 5 | intron | mRNA splicing defect? | Kilinc et al. (NL#70) |
| | | 19 | | |
| 3850 − 3T − >G | T to G at 3850 − 3 | intron | mRNA splicing defect | Dörk et al. 1993a |
| | | 19 | | |
| 3850 − 1G − >A | G to A at 3850 − 1 | intron | mRNA splicing defect | Audrézet et al. 1993a |
| | | 19 | | |
| V1240G | T to G at 3851 | 20 | Val to Gly at 1240 | Zielenski et al. 1999* |
| G1244V | G to T at 3863 | 20 | Gly to Val at 1244 | Savov et al. 1994b |
| G1244E | G to A at 3863 | 20 | Gly to Glu at 1244 | Devoto et al. 1991 |
| T1246I | C to T at 3869 | 20 | Thr to Ile at 1246 | Férec et al. (NL#64) |
| | | | (mutation?) | |
| G1247R | G to A at 3871 | 20 | Gly to Arg at 1247 | Casals et al. (NL#69) |
| G1249R | G to A at 3877 | 20 | Gly to Arg at 1249 | Dijkstra et al. 1994 |
| G1249E | G to A at 3878 | 20 | Gly to Glu at 1249 | Greil et al. 1994 |
| S1251N | G to A 3884 | 20 | Ser to Asn at 1251 | Kälin et al. 1992a; |
| | | | | Mercier et al. 1993a |
| T1252P | A to C at 3886 | 20 | Thr to Pro at 1252 | Wallace (NL#69) |
| S1255P | T to C at 3895 | 20 | Ser to Pro at 1255 | Lissens et al. 1992 |
| S1255L | C to T at 3896 | 20 | Ser to Leu at 1255 | Bienvenu et al. (NL#69) |
| F1257L | T to G at 3903 | 20 | Phe to Leu at 1257 | Férec 1998* |
| delta L1260 | deletion of ACT from either | 20 | deletion of Leu at 1260 or | Hermans et al. 1994 |
| | 3909 or 3912 | | 1261 | |
| 3922del10 − >C | deletion of 10 bp from 3922 | 20 | deletion of Glu1264 to | Schwarz et al.(NL#69) |
| | and replacement with 3921 | | Glu1266 | |
| I1269N | T to A at 3938 | 20 | Ile to Asn at 1269 | McDowell et al. (NL#66) |
| D1270N | G to A at 3940 | 20 | Asp to Asn at 1270 | Dean et al. 1991 |
| W1282G | T to G at 3976 | 20 | Trp to Gly at 1282 | Faucz et al. (NL#69) |
| W1282R | T to C at 3976 | 20 | Trp to Arg at 1282 | Ivaschenko et al. 1993 |
| W1282C | G to T at 3978 | 20 | Trp to Cys at 1282 | Férec et al. (NL#69) |
| R1283M | G to T at 3980 | 20 | Arg to Met al 1283 | Cheadle et al. 1992 |
| R1283K | G to A at 3980 | 20 | Arg to Lys at 1283 | Chevalier & Bozon |
| | | | | (NL#54) |
| F1286S | T to C at 3989 | 20 | Phe to Ser at 1286 | Dorval et al. 1993 |
| Q1291R | A to G at 4004 | 20 | Gln to Arg at 1291 | Dörk et al. 1994b |
| Q1291H | G to C at 4005 | 20 | Gln to His at 1291; | Jones et al. 1992 |
| | | | mRNA splicing defect (?) | |
| 4005 + 1G − >A | G to A at 4005 + 1 | intron | mRNA splicing defect | Férec et al. 1992 |
| | | 20 | | |
| 4005 + 2T − >C | T to C at 4005 + 2 | intron | mRNA splicing defect | Boman (NL#69) |
| | | 20 | | |
| 4006 − 61del14 | deletion of 14 bp from 4006 − 61 | intron | mRNA splicing defect? | Friedman et al. (NL#59) |
| | to 4006 − 47 | 20 | | |
| 4006 − 19del3 | deletion of 3 bp from 4006 − 19 | intron | mRNA splicing defect? | Naseem et al. (NL#36) |
| | | 20 | | |
| 4006 − 14C − >G | C to G at 4006 − 14 | intron | mRNA splicing defect? | Poncin (NL#69) |
| | | 20 | | |
| 4006 − 8T − >A | T to A at 4006 − 8 | intron | mRNA splicing defect? | Chevalier-Porst & Bozon |
| | | 20 | | (NL#70) |
| 4006 − 4A − >G | A to G at 4006 − 4 | intron | mRNA splicing defect? | Chomel et al. (NL#68) |
| V1293I | G to A at 4009 | 21 | Val to Ile at 1293 | Férec et al. (NL#69) |
| T1299I | C to T at 4028 | 21 | Thr to Ile at 1299 | Liechti-Gallati (NL#68) |
| F1300L | T to C at 4030 | 21 | Phe to Leu at 1300 | Poncin (NL#69) |
| N1303H | A to C at 4039 | 21 | Asn to His at 1303 | Claustres et al. 1992b |
| N1303I | A to T at 4040 | 21 | Asn to Ile at 1303 | Lissens et al. (NL#66); |
| | | | | Férec et al. (NL#66) |
| N1303K | C to G at 4041 | 21 | Asn to Lys at 1303 | Osborne et al. 1991 |
| D1305E | T to A at 4047 | 21 | Asp to Glu at 1305 | Claustres et al. (NL#69) |
| Q1313K | C to A at 4069 | 21 | Gln to Lys at 1313 | Malone et al. (NL#68) |
| V1318A | T to C at 4085 | 21 | Val to Ala at 1318 | Férec 1998* |
| E1321Q | G to C at 4093 | 21 | Glu to Gln at 1321 | Férec et al. (NL#64) |
| 4096 − 28G − >A | G to A at 4096 − 28 | intron | mRNA splicing defect? | Claustres et al. (NL#68) |
| | | 21 | | |
| 4096 − 3C − >G | C to G at 4096 − 3 | intron | mRNA splicing defect? | Claustres et al. (NL#69) |
| | | 21 | | |
| L1335P | T to C at 4136 | 22 | Leu to Pro at 1335 | Zielenski et al. (NL#70) |
| F1337V | T to G at 4138 | 22 | Phe to Val at 1337 | Scheffer et al. (NL#70) |
| | | | (CBAVD) | |
| L1339F | C to T at 4147 | 22 | Leu to Phe at 1339 | Girodon et al. 1999* |
| G1349S | G to A at 4177 | 22 | Gly to Ser at 1349 | Yoshimura 1999* |
| G1349D | G to A at 4178 | 22 | Gly to Asp at 1349 | Beaudet et al. 1991 |
| K1351E | A to G at 4183 | 22 | Lys to Glu at 1351 | Dörk et al. (NL#69) |
| | | | (CBAVD) | |
| Q1352H* | G to C at 4188 | 22 | Gln to His at 1352 | Nukiwa & Seyama |
| | | | | (NL#55) |
| R1358S | A to T at 2406 | 22 | Arg to Ser at 1358 | Férec 1999* |
| A1364V | C to T at 4223 | 22 | Ala to Val at 1364 | Claustres et al (NL#67) |
| | | | CBAVD | |
| D1377H | G to C at 4261 | 22 | Asp to His at 1377 | Costes et al. (NL#56) |
| L1388Q | T to A at 4295 | 23 | Leu to Gln at 1388 | Dörk et al. (NL#68) |
| | | | (CBAVD) | |
| V1397E | T to A at 4322 | 23 | Val to Glu at 1397 | Petreska et al. 1994 |
| E1409V | A to T at 4358 | 23 | Glu to Val at 1409 | Claustres et al. (NL#55) |
| Q1412X | C to T at 4366 | 23 | Gln to Stop at 1412 | Wallace & Tassabehji |
| | | | | (NL#60) |
| 4374 + 10T − >C | T to C at 4374 + 10 | intron | splicing? | Férec 1998* |
| | | 23 | | |
| 4374 + 1G − >A | G to A at 4374 + 1 | intron | mRNA splicing defect | Fanen et al. 1992 |
| | | 23 | | |
| 4374 + 1G − >T | G to T at 4374 + 1 | intron | mRNA splicing defect | Dörk et al.(NL#38) |
| | | 23 | | |
| 4375 − 1G − >C | G to C at 4375 − 1 | intron | splicing mutation | Chevalier-Porst & Bozon |
| | | 23 | | 1999* |
| R1422W | C to T at 4396 | 24 | Arg to Trp at 1422 | Claustres et al. (NL#70) |
| S1426P | T to C at 4408 | 24 | Ser to Pro at 1426 | Férec 1999* |
| D1445N | G to A at 4465 | 24 | Asp to Asn at 1445 | Antoniadi et al. (NL#69) |
| R1453W | C to T at 4489 | 24 | Arg to Trp at 1453 | Yoshimura 1999* |
| CFTRdele14a | deletion of >= 1.2 kb including | 14a | aberrant mRNA splicing | Egan et al. (NL#68) |
| | exon 14a | | | |
| CFTRdele19 | deletion of 5.3 kb, removing | 19 | ? | Girodon et al. 1999 |
| | exon 19 | | | |
| 2104insA + 2109 − | insertion of A at 2104, deletion | 13 | ? | Girodon et al. 1999* |
| 2118del10 | of 10 bp at 2109 | | | |
| CF25kbdel | Complex | intron 3 | ? | Shackleton et al. (NL# |
| | deletion/rearrangement | | | 70) |

The above disclosure generally describes the present invention. A more complete understanding can be obtained by reference to the following specific examples which are provided herein for purposes of illustration only, and are not intended to limit the scope of the invention.

### EXAMPLES

Development of a Polypeptide that Exerts Only an Activating Effect on CFTR

The activating peptide of Q4N2NEG2 was created by substituting glutamine residues for glutamic acid residues at four sites and asparagines for aspartic acid residues at two sites of the authentic NEG2 peptide sequence GLEISFEINEEDLKECFFDDME (SEQ ID NO: 7). In addition, a serine residue was substituted for cysteine, to prevent peptide dimerization, and norleucine was substituted for methionine, to prevent oxidation. These changes create a peptide with reduced chemical reactivity and high predicted helical structure, confirmed by circular dichroism, as well as reduced net negative charge (from −9 to −3). Attempts to eliminate negative charge completely resulted in an insoluble peptide. When this peptide was added to the cis (intracellular) side of CFTR channels captured in the planar lipid bilayer, at concentration ranging 0.5 to 14 μM, marked dose-related stimulation of channel activity was observed. At concentrations of 4-6 μM Po of CFTR doubles. No inhibitory activity was seen in any experiment at any concentration of peptide.

Q4N2NEG2 Polypeptide Stimulates Wild-type CFTR Protein.

To test whether the Q4N2NEG2 polypeptide is responsible for increasing the open probability of the CFTR channel, synthetic Q4N2NEG2, a 22 amino acid peptide, was added to the cis-intracellular side of single CFTR channels captured in the planar lipid bilayer (FIG. 1). The diary plot of open probability as a function of time shows the activity of a single wt-CFTR channel during the course of the experiment (FIG. 1A). During stimulation, the open probability doubles and more transitions are observed between the open and closed states (FIG. 1B). The open probability observed in 5 experiments at 4 μM concentration Q4N2NEG2 is shown to be increased by about two-fold in the graph (FIG. 1C).

Q4N2NEG2 Polypeptide Stimulates Mutant G551D CFTR Protein.

The Q4 N2 NEG2 peptide sequence has been tested on one mutant form of CFTR, G551D, which reaches the plasma membrane. In the planar lipid bilayer, Q4N2NEG2 increased the open probability of G551 by about threefold. Thus, this peptide is useful to stimulate channel activity in mutant forms of CFTR that reach the plasma membrane.

The NEG2 Polypeptide can be Rendered Inhibitory to CFTR

The NEG2 sequence can also be rendered inhibitory, with no stimulatory activity, by scrambling the sequence such that the resulting peptide is predicted to not have helical tendencies, as confirmed by circular dichroism measurements, but retains the full net negative charge of −9. This peptide, called scrambled NEG2, inhibits channel activity by about 90% at 6 μM concentration, with no stimulation observed at any concentration. In addition, insertion of a proline residue into the middle of the NEG2 sequence also results in a peptide which inhibits channel activity by about 60%, but does not stimulate. Proline residues are known to disrupt helical structures.

Methods Used In Examples

Subcloning of CFTR Gene

The wt CFTR cDNA was subcloned into an Epstein-Barr virus-based episomal eukaryotic expression vector, pCEP4 (Invitrogen, San Diego, Calif.), between the Nhe1 and Xho1 restriction sites.

Expression of CFTR in HEK 293 Cells

A human embryonic kidney cell line (293-EBNA HEK; Invitrogen) was used for transfection and expression of the CFTR proteins (Ma et al., 1997, Ma et al., 1996, Xie et al., 1995). The HEK-293 cell line contains a pCMV-EBNA vector, which constitutively expresses the Epstein-Barr virus nuclear antigen-1 (EBNA-1) gene product and increases the transfection efficiency of Epstein-Barr virus-based vectors. The cells were maintained in Dulbecco's Modified Eagle Medium with 10% FBS and 1% L-glutamine. Geneticin (G418, 250 (g/ml) was added to the cell culture medium to maintain selection of the cells containing the pCMV-EBNA vector. Lipofectamine reagent (Life Technologies, Inc) in Optimem media (serum-free) was used to transfect the HEK-293 cells with pCEP4(wt). After 5 hours, serum was added to the media (10% final serum concentration). Twenty-four hours after transfection, the transfection media was replaced with fresh media. The cells were harvested two days after transfection and microsomal membrane vesicles were prepared for single channel measurements in the lipid bilayer reconstitution system.

Vesicle Preparation from Transfected HEK 293 Cells

HEK-293 cells transfected with pCEP4(CFTR) were harvested and homogenized using a combination of hypotonic lysis and Dounce homogenization in the presence of protease inhibitors (Ma et al., 1997, Ma et al., 1996, Xie et al., 1995). Microsomes were collected by centrifugation of postnuclear supernatant (4500×g, 15 min) at 100,000×g for 20 min and resuspended in a buffer containing 250 mM sucrose, 10 mM HEPES, pH 7.2. The membrane vesicles were stored at −75° C. until use.

Reconstitution of CFTR Channels in Lipid Bilayer Membranes

Lipid bilayer membranes were formed across an aperture of ˜200 (m diameter with a mixture of phosphatidylethanolamine:phosphatidylserine:cholesterol in a ratio of 5:5:1. The lipids were dissolved in decane at a concentration of 33 mg/ml. The recording solutions contained: cis (intracellular), 200 mM CsCl, 1 mM MgCl₂, 2 mM ATP, and 10 mM HEPES-Tris (pH 7.4); trans (extracellular), 50 mM CsCl, 10 mM HEPES-Tris (pH 7.4). Vesicles (1-4 (1) containing wild-type CFTR were added to the cis solution. The PKA catalytic subunit was present at a concentration of 50 units/ml in the cis solution unless noted otherwise. Single channel currents were recorded with an Axopatch 200A patch clamp unit (Axon Instruments). The currents were sampled at 1-2.5 ms/point. Single channel data analyses were performed with pClamp and TIPS softwares.

### REFERENCES

Anderson, M. P., Berger, H. A., Rich, D. P., Gregory, R. J., Smith, A. E., and Welsh, M. J. (1991). Nucleoside triphosphates are required to open the CFTR chloride channel. Cell 67, 775-784.

Bear, C. E., Li, C., Kartner, N., Bridges, R. J., Jensen, T. J., Ramjeesingh, M., and Riordan, J. R. (1992). Purification and functional reconstitution of the cystic fibrosis transmembrane conductance regulator (CFTR). Cell 68, 809-818.

Carson, M. R., Travis, S. M., and Welsh, M. J. (1995). The two nucleotide-binding domains of cystic fibrosis transmembrane conductance regulator (CFTR) have distinct functions in controlling channel activity. J. Biol. Chem. 270, 1711-1717.

Cheng, S. H., Rich, D. P., Marshall, J., Gregory, R. J., Welsh, M. J., and Smith, A. E. (1991). Phosphorylation of the R domain by cAMP-dependent protein kinase regulates the CFTR chloride channel. Cell 66, 1027-1036.

Cotten, J. F. and Welsh, M. J. (1997). Covalent modification of the regulatory domain irreversibly stimulates cystic fibrosis transmembrane conductance regulator. J. Biol. Chem. 272, 25617-25622.

Dulhanty, A. M. and Riordan, J. R. (1994). Phosphorylation by cAMP-dependent protein kinase causes a conformational change in the R domain of the cystic fibrosis transmembrane conductance regulator. Biochemistry 22, 4072-4079.

Gadsby, D. C. and Nairn, A. C. (1994). Regulation of CFTR channel gating. Trends Biochem. Sci. 19, 513-518.

Geourjon, C. and Deleage, G. (1995). SOPMA: significant improvements in protein secondary structure prediction by consensus prediction from multiple alignments. CABIOS 11, 681-684.

Gunderson, K. L. and Kopito, R. R. (1995). Conformational states of CFTR associated with channel gating: the role of ATP binding and hydrolysis. Cell 82, 231-239.

Higgens, C. F. (1992). ABC transporters: from microorganisms to man. Annu. Rev. Cell Biol. 8, 67-113.

Ma, J. and Davis, P. B. (1998). What we know and what we do not know about cystic fibrosis transmembrane conductance regulator. Clinics in Chest Medicine 19, 459-471.

Ma, J., Tasch, J. E., Tao, T., Zhao, J., Xie, J., Drumm, M. L., and Davis, P. B. (1996). Phosphorylation-dependent block of cystic fibrosis transmembrane conductance regulator chloride channel by exogenous R domain protein. J. Biol. Chem. 271, 7351-7356.

Ma, J., Zhao, J., Drumm, M. L., Xie, J., and Davis, P. B. (1997). Function of the R domain in the cystic fibrosis transmembrane conductance regulator chloride channel. J. Biol. Chem. 272, 28133-28141.

Picciotto, M. R., Cohn, J. A., Bertuzzi, G., Greengard, P., and Nairn, A. C. (1992). Phosphorylation of the cystic fibrosis transmembrane conductance regulator. J. Biol. Chem. 267, 12742-12752.

Quinton, P. M. (1986). Missing Cl⁻ conductance in cystic fibrosis. Am. J. Physiol. 251, C649-C652.

Rich, D. P., Berger, H. A., Cheng, S. H., Travis, S. M., Saxena, M., Smith, A. E., and Welsh, M. J. (1993). Regulation of the cystic fibrosis transmembrane conductance regulator Cl⁻ channel by negative charge in the R domain. J. Biol. Chem. 268, 20259-20267.

Rich, D. P., Gregory, R. J., Anderson, M. P., Manavalan, P., Smith, A. E., and Welsh, M. J. (1991). Effect of deleting the R domain on CFTR-generated chloride channels. Science 253, 205-207.

Riordan, J., Rommens, J., Kerem, B.-S., Noa, A., Rozmahel, R., Grzelczak, Z., Zielenski, J., Lok, S., Plavsic, N., Chou, J.-L., Drumm, M., Iannuzzi, M., Collins, F., and Tsui, L.-C. (1989). Identification of the cystic fibrosis gene: cloning and characterization of complementary DNA. Science 245, 1066-1073.

Rost, B. and Sander, C. (1993). Prediction of protein structure at better than 70% accuracy. J. Mol. Biol. 232, 584-599.

Rost, B. and Sander, C. (1994). Combining evolutionary information and neural networks to predict protein secondary structure. Proteins 19, 55-72.

Tabcharani, J. A., Chang, X.-B., Riordan, J. R. and Hanrahan, J. W. (1991). Phosphorylation-regulated C− channel in CHO cells stably expressing the cystic fibrosis gene. Nature 352, 628-631.

Tao, T., Xie, J., Drumm, M. L., Zhao, J., Davis, P. B., and Ma, J. (1996). Slow conversions among subconductance states of cystic fibrosis transmembrane conductance regulator chloride channel. Biophys. J. 70, 743-753.

Vankeerberghen, A., Wei, L., Jaspers, M., Cassiman, J.-J., Nilius, B., and Cuppens, H. (1998). Characterization of 19 disease-associated missense mutations in the regulatory domain of the cystic fibrosis transmembrane conductance regulator. Hum. Mol. Genet. 7, 1761-1769.

Welsh, M. J. and Smith, A. E. (1993). Molecular mechanisms of CFTR chloride channel dysfunction in cystic fibrosis. Cell 73, 1251-1254.

Winter, M. C. and Welsh, M. J. (1997). Stimulation of CFTR activity by its phosphorylated R domain. Nature 389, 294-296.

Xie, J., Drumm, M. L., Ma, J., and Davis, P. B. (1995). Intracellular loop between transmembrane segments IV and V of cystic fibrosis transmembrane conductance regulator is involved in regulation of chloride channel conductance state. J. Biol. Chem. 270, 28084-28091.

Zielenski, J. and Tsui, L. C. (1995). Cystic fibrosis: genotypic and phenotypic variations. Annu. Rev. Genetics 29, 777-807.

1. A polypeptide comprising an amino acid sequence of SEQ ID NO: 6, wherein the polypeptide retains a net negative charge of 1-8.

2. The polypeptide of claim 1 wherein the polypeptide retains a net negative charge of 2-8.

3. The polypeptide of claim 1 wherein the polypeptide retains a net negative charge of 3-8.

4. The polypeptide of claim 1 wherein the polypeptide retains a net negative charge of 4-8.

5. The polypeptide of claim 1 wherein the polypeptide retains a net negative charge of 5-8.

6. The polypeptide of claim 1 wherein the polypeptide retains a net negative charge of 6-8.

7. The polypeptide of claim 1 wherein the polypeptide retains a net negative charge of 7-8.

8. The polypeptide of claim 1 wherein amino acid residue sixteen is serine.

9. The polypeptide of claim 1 wherein amino acid residue twenty-one is norleucine.

10. The polypeptide of claim 1 which comprises the amino acid sequence of SEQ ID NO: 1.

11. A composition comprising the polypeptide of claim 1 and a pharmaceutically acceptable carrier.

12. The polypeptide of claim 1 consisting of the sequence of SEQ ID NO: 1.

13. The polypeptide of claim 1 wherein the polypeptide is fused to a membrane-penetrating peptide.

14. The polypeptide of claim 13 wherein the membrane-penetrating peptide is selected from the group consisting of: VP-22 (SEQ ID NO: 3), (SEQ ID NO: 4), and (SEQ ID NO: 5).

15. A method of activating a CFTR protein comprising:
administering an effective amount of a polypeptide to a cell comprising a CFTR protein which forms a cAMP-regulated chloride channel, said polypeptide comprising the sequence of SEQ ID NO: 6, whereby the CFTR protein is activated.

16. The method of claim 15 wherein the polypeptide comprises the sequence of SEQ ID NO: 1.

17. The method of claim 15 wherein the effective amount of the polypeptide increases open probability of the channel formed by the CFTR by at least 25%.

18. The method of claim 15 wherein open probability of the channel formed by the CFTR increases by at least 50%.

19. The method of claim 15 wherein open probability of the channel formed by the CFTR increases by at least 75%.

20. The method of claim 15 wherein open probability of the channel formed by the CFTR increases by at least 100%.

21. The method of claim 15 wherein open probability of the channel formed by the CFTR increases by at least 125%.

22. The method of claim 15 wherein open probability of the channel formed by the CFTR increases by at least 150%.

23. The method of claim 15 wherein open probability of the channel formed by the CFTR increases by at least 175%.

24. The method of claim 15 wherein open probability of the channel formed by the CFTR increases by at least 200%.

25. The method of claim 15 wherein open probability of the channel formed by the CFTR increases by at least 300%.

26. The method of claim 15 wherein said polypeptide is administered to achieve a concentration of 0.5 to 14 μM.

27. The method of claim 15 wherein said polypeptide is administered to achieve a concentration of 4-6 μM.

28. The method of claim 15 wherein the CFTR protein is a mutant which reaches the cell's plasma membrane but fails to undergo full activation in the absence of said polypeptide.

29. The method of claim 28 wherein the mutant CFTR protein is selected from the group consisting of −816C→T, −741T→G, −471delAGG, −363C/T, −102T→A, −94G→T, −33G→A 132C→G, P5L, S10R, S13F, 185+1G→T, 185+4A→T, 186−13C→G, W19C, G27E, R31C, R31L, 232del18, S42F, D44G, A46D, 279A/G, I50T, S50P, S50Y, 296+3insT, 296+1G→T, 296+1G→C, 296+2T→C, 296+9A→T, 296+12T→C, 297−28insA, 297−3C→A, 297−3C−×T, 297−2A→G, 297−10T→G, 297−12insA, E56K, W57G, W57R, D58N, D58G, E60K, E60L, N66S, P67L, K68E, K68N, A72T, A72D, R74W, R74Q, R75L, W79R, G85E, G85V, F87L, L88S, Y89C, L90S, G91R, 405+1G→A, 405+3A→C, 405+4A→G, 406−10C→G, 406−6T→C, 406−3T→C, 406−2A→G, 406−2A→C, 406−1G→C, 406−1G→A, 406−1G→T, E92K, A96E, Q98R, P99L, I105N, S108F, Y109N, Y109C, D110H, D110Y, D110E, P111A, P111L, delta E115, E116Q, E116K, R117C, R117H, R117P, R117L, A120T, I125T, G126D, L137R, L137H, L138ins, H139R, P140S, P140L, A141D, H146R, I148T, I148N, G149R, M152V, M152R, 591del18, A155P, S158R, Y161N, Y161D, Y161S, K162E, 621G→A, 621+1G→T, 621+T→C, 621+2T→G, 621+3A→G, 622−2A→C, 622−1G→A, L165S, K166Q, R170C, R170G, R170H, I175V, I177T, G178R, Q179K, N186K, N187K, D192N, delta D192, D192G, E193K, 711+1G→T, 711+3A→C, 711+3A→G, 711+3A→T, 711+5G→A, 711+34A→G, 712−1G→T, G194V, A198P, H199Y, H199Q, V201M, P205S, L206W, L206F, A209S, E217G, Q220R, C225R, L227R, V232D, Q237E, G239R, G241R, M243L, M244K, R248T, 875+1G→C, 875+1G→A, 876−14del12, 876−10del8, 876−3C→T, R258G, V920L, M265R, E278del, N287Y, 994del9, 1002−3T→G, E292K, R297W, R297Q, A299T, Y301C, S307N, A309D, A309G, delta F311, F311L, G314R, G314V, G314E, F316L, V317A, L320V, L320F, V322A, L327R, R334W, R334L, R334Q, I336K, T338I, E474K, L346P, R347C, R347H, R347P, R347L, M348K, A349V, R352W, R352Q, Q353H, Q359K/T360K, Q359R, W361R(T→C), W361R(T→A), S364P, L365P, 1243ins6, 1248+1G→A, 1249−29delAT, 1249−27delTA, 1249−5A→G, L375F, E379X, L383S, T360R, V392A, V392G, M394R, A399V, E403D, 1341G→A, 1341G→A, 1341+1G→A, 1341+18A→C, 1342−11TTT→G, 1342−2A→C, 1342−1G→C, E407V, N418S, G424S, D443Y, I444S, Q452P, delta L453, A455E, V456F, G458V, 1524+6insC, 1525−1G→A, S466L, G480S, G480C, G480D, H484Y, H484R, S485C, C491R, S492F, Q493R, P499A, T501A, I502T, E504Q, I506L, delta I507, I506S, I506T, delta F508, F508S, D513G, Y517C, V520F, V520I, 1706del16, 1706del17, E527Q, E527G, 1716−1G→A, E528D, 1716+2T→C, 1717−8G→A, 1717−3T→G, 1717−2A→G, 1717−1G→A, 1717−9T→A, D529H, A534E, I539T, G544S, G544V, S549R(A→C), S549N, S549I, S549R(T→G), G550R, G551S, G551D, Q552K, R553G, R553Q, R555G, I556V, L558S, A559T, A559E, R560K, R560T, 1811+1G→C, 1811+1.6kbA→G, 1811+18G→A, 1812−1G→A, R560S, A561E, V562L, V562I, Y563D, Y563N, Y563C, L568F, Y569D, Y569H, Y569C, L571S, D572N, P574H, G576A, Y577F, D579Y, D579G, D579A, T582I, T582R, S589N, S589I, 1898+1G→T, 1898+1G→C, 1898+1G→A, 1898+3A→C, 1898+3A→G, 1898+5G→T, 1898+5G→A, 1898+73T→G, R600G, I601F, V603F, T604I, 1949del84, H609R, L610S, A613T, D614Y, D614G, I618T, L619S, H620P, H620Q, G622D, G628R(G→A), G628R(G→C), L633P, L636P, D648V, D651N, T665S, E672del, K683R, F693L(CTT), F693L(TTG), K698R, E725K, P750L, V754M, T760M, R766M, N782K, R792G, A800G, E822K, E826K, 2622+1G→T, 2622+1G→A, 2622+2del6, D836Y, R851L, C866Y, L867X, 2751G→A, 2751+2T→A, 2751+3A→G, 2752−26A→G, 2752−1G→T, 2752−1G→C, T908N, 2789+2insA, 2789+3delG, 2789+5G→A, 2790−2A→G, 2790−1G→C, 2790−1G→T, Q890R, D891G, S895T, T896I, N900T, 2851A/G, S912L, Y913C, Y917D, Y917C, I918M, Y919C, V920M, D924N, L927P, F932S, R933S, V938G, H939D, H939R, S945L, S945L, K946X, H949Y, H949R, M952T, M952I, M961I, L967S, G970R, 3040+2T→C, 3041−1G→A, G970D, L973F, L973P, S977P, S977F, D979V, D979A, I980K, D985H, D985Y, I991V, D993Y, F994C, 3120G→A, 3120+1G→A, 3121−2A→T, 3121−2A→G, 3121−1G→A, L997F, 3131del15, I1005R, A1006E, V1008D, A1009T, P1013L, Y1014C, P1021S, 3195del6, 3196del54, 3199del6, I1027T, M1028R, M1028I, Y1032C, I1366T, 3271delGG, 3271+1G→A, 3271+1delGG, 3272−26A→G, 3272−9A→T, 3272−4A→G, 3272−1G→A, G1047D, F1052V, T1053I, T1053I, H1054D, T1057A, K1060T, G1061R, L1065F, L1065R, L1065P, R1066S, R1066C, R1066H, R1066L, A1067T, A1067D, G1069R, R1070W, R1070Q, R1070P, Q1071P, Q1071H, P1072L, F1074L, L1077P, H1085R, T1086I, N1088D, Y1082H, L1093P, L1096R, W1098R, Q1100P, M1101R, M1101K, S1118F, S1118C, G1123R, 3499+2T→C, 3499+3A→G, 3499+6A→G, 3500−2A→G, E1123del, G1127E, 3523A→G, A1136T, M1137V, M1137R, I1139V, delta M1140, M1140K, T1142I, V1147I, N1148K, D1152H, V1153E, D1154G, 3600G→A, 3600+2insT, 3600+5G→A, 3601−20T→C, 3601−17T→C, 3601−2A→G, S1159P, S1159F, D1168G, K1177R, 3696G/A, V1190P, 3750delAG, 3755delG, M1210I, V1212I, L1227S, E1228G, I1230T, I1234V, S1235R, G1237S, Q1238R, 3849G→A, 3849+1G→A, 3849+4A→G, 3849+10kbC→T, 3849+5G→A, 3850−3T→G, 3850−1G→A, V1240G, G1244V, G1244E, T1246I, G1247R, G1249R, G1249E, S1251N, T1252P, S1255P, S1255L, F1257L, delta L1260, 3922del10→C, I1269N, D1270N, W1282G, W1282R, W1282C, R1283M, R1283K, F1286S, Q1291R, Q1291H, 4005+1G→A, 4005+2T→C, 4006−61del14, 4006−19del3, 4006−14C→G, 4006−8T→A, 4006−4A→G, V1293I, T12991, F1300L, N1303H, N1303I, N1303K, D1305E, Q1313K, V1318A, E1321Q, 4096−28G→A, 4096−3C→G, L1335P, F1337V, L1339F, G1349S, G1349D, K1351E, Q1352H*, R1358S, A1364V, D1377H, L1388Q, V1397E, E1409V, Q1412X, 4374+10T→C, 4374+1G→A, 4374+1G→T, 4375−1G→C, R1422W, S1426P, D1445N, R1453W, CFTRdele14a, CFTRdele19, 2104insA+2109−2118del10, and CF25kbdel as listed in Table 1.

30. The method of claim 15 wherein the polypeptide is administered in an aerosol to a patient with a mutant CFTR protein.

31. The method of claim 15 wherein the polypeptide is administered in an aerosol to a patient with insufficient amounts of wild-type CFTR to maintain chloride transport.

32. The method of claim 30 wherein the aerosolized polypeptide is co-administered with an expression vector wherein said expression vector encodes wild-type CFTR protein.

33. The method of claim 31 wherein the aerosolized polypeptide is co-administered with an expression vector wherein said expression vector encodes wild-type CFTR protein.

34. A method of activating a CFTR protein comprising:
applying an effective amount of a polypeptide to a CFTR protein in a lipid bilayer wherein the polypeptide comprises the amino acid sequence of SEQ ID NO: 6, whereby the CFTR protein is activated.

35. The method of claim 34 wherein the polypeptide comprises the amino acid sequence of SEQ ID NO: 1.

36. The method of claim 34 further comprising measuring a change in conductance upon applying the polypeptide.

37. A method of synthesizing a CFTR activating polypeptide comprising:
sequentially linking units of one or more amino acid residues to form a polypeptide comprising the amino acid sequence of SEQ ID NO: 6.

38. The method of claim 37 wherein F-moc synthesis is used.

39. The method of claim 37 wherein the polypeptide has the sequence of SEQ ID NO: 1.

40. A polypeptide comprising the amino acid sequence as shown in SEQ ID NO: 2.

41. The polypeptide of claim 40 wherein the polypeptide is fused to a membrane-penetrating peptide.

42. The polypeptide of claim 41 wherein the membrane-penetrating peptide is selected from the group consisting of: VP-22 (SEQ ID NO: 3), (SEQ ID NO: 4) and (SEQ ID NO: 5).

43. A nucleic acid molecule comprising a nucleotide sequence encoding a polypeptide according to SEQ ID NO: 2.

44. A method of activating a CFTR protein, comprising:
administering a nucleic acid comprising a sequence encoding a polypeptide according to SEQ ID NO: 2 to a cell comprising the CFTR protein, whereby the polypeptide is expressed and the CFTR protein is activated.

45. The method of claim 44 wherein the cell is in a patient and the nucleic acid is administered as an aerosol to the patient's airways.

46. The method of claim 45 wherein the nucleic acid molecule is co-administered with an expression vector encoding a wild-type CFTR protein.


SEQUENCE LISTING (7 sequences)

SEQ ID NO: 1 - protein, 22 aa, homo sapiens
  MOD_RES (1)..(1): ACETYLATION
GLEISEQINQQNLKQSFFNDLE

SEQ ID NO: 2 - protein, 22 aa, homo sapiens
GLEISEQINQQNLKQSFFNDME

SEQ ID NO: 3 - protein, 301 aa, homo sapiens
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVATKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 4 - protein, 27 aa, homo sapiens
GWTLNSAGYLLGKINLKALAALAKKIL

SEQ ID NO: 5 - protein, 16 aa, homo sapiens
RQIKIWFQNRRMKWKK

SEQ ID NO: 6 - protein, 22 aa, homo sapiens
  CDS (1)..(11): Xaa=Glu or Gln
GLXISXXINXXXLKXXFFXXXX

SEQ ID NO: 7 - protein, 22 aa, homo sapiens
GLEISEEINEEDLKECFFDDME